(19) United States
(12) Patent Application Publication
(10) Pub. No.: US 2008/0254033 A1
(43) Pub. Date: Oct. 16, 2008

(54) Pharmaceutical Compositions Containing Antagonists to Lrp4, Lrp8 or Megalin for Treatment of Diseases

(76) Inventors: Kristen Pierce, Burlingame, CA (US); Amy L.Tsui Collins, Oakland, CA (US); Hongbing Zhang, Albany, CA (US); Lewis Thomas Williams, Mill Valley, CA (US); Pierre Alvaro Beaurang, Greenbrae, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/586,669

(22) PCT Filed: Jan. 21, 2005
(86) PCT No.: PCT/US05/01883

§ 371 Date: Oct. 2, 2007

Related U.S. Application Data
(60) Provisional application No. 60/538,322, filed on Jan. 21, 2004.

(51) Int. Cl. A61K 39/395; A61P 25/28; C07K 14/435; C12N 15/85; C07K 16/00; C12N 5/10; C12N 15/12; A61K 31/7105

(52) U.S. Cl. 424/139.1; 530/324; 530/326; 530/387.9; 536/23.5; 435/325

(57) ABSTRACT

The present invention provides LRP4, LRP8 and LRP2 (megalin) polypeptides; polynucleotides encoding such; modulators of LRP4, LRP8 and LRP2 activity; and pharmaceutical compositions for the treatment of diseases. Compositions of the invention contain a pharmaceutically acceptable carrier or excipient and at least one modulator that is capable of binding to or interfering with the activity of LRP4, LRP8, LRP2 and active fragments thereof. The invention provides modulators, such as antibodies, RNAi molecules, anti-sense molecules and ribozymes. Additionally, the invention includes methods for the treatment of diseases, such as proliferative diseases and degenerative diseases, and methods of administration of the compositions of the invention.

### PRIORITY CLAIM

This application claims the benefit of provisional application 60/538,322, filed in the United States Patent and Trademark Office on Jan. 21, 2004, the disclosure of which is hereby incorporated by reference.

### DESCRIPTION OF THE INVENTION

### Technical Field

The present invention relates to pharmaceutical compositions for the treatment of diseases. Compositions of the invention may contain a pharmaceutically acceptable carrier or excipient and at least one modulator that will bind to or interfere with the activity of megalin (hereinafter referred to as “LRP2”), LRP4, LRP8 and active fragments thereof. The invention further provides modulators, such as antibodies, RNAi molecules, anti-sense molecules and ribozymes. Additionally, the invention includes methods of treatment of diseases, such as proliferative and degenerative diseases, and methods of administration of the compositions of the invention.

### BACKGROUND OF THE INVENTION

The LDL receptor-related protein (LRP) is larger than, but structurally similar to, other members of the LDL receptor gene family, a family of endocytic receptors (Willnow et al., Nat. Cell Biol., 1:E157 (1999); Herz et al., Nat. Rev. Neurosci., 1:51 (2000)). Whereas the LDL receptor, the first-discovered member of this family, appears to act solely in lipoprotein metabolism, LRP and some known members of this family appear to have other distinct functions.

Known LRPs, like many members of the LDL receptor gene family, contain five common structural units shown that typically include, for example, ligand-binding (complement) type cysteine-rich repeats, epidermal growth factor (EGF) receptor-like cysteine-rich repeats, YWTD domains, a single membrane-spanning segment, and a cytoplasmic tail that contains between one and three NPxY motifs. Ligand-binding-type repeats in LRP occur in clusters containing between two and eleven individual repeats. Examples of known ligands are listed, for example, in Herz et al., J. Clin. Invest., 108:779 (2001) (Table 1). Many of the known ligands for LRP, for which the binding sites have been mapped, interact with these ligand-binding-type domains (Neels et al., J. Biol. Chem., 274:31305 (1999)). These are followed by EGF precursor homology domains, which consist of the two EGF repeats, six YWTD repeats that are arranged in a propeller-like structure (Springer, T. A., J. Mol. Biol., 283:837 (1998)), and another EGF repeat. Six EGF repeats precede the single membrane-spanning segment. The cytoplasmic tail contains two NPxY motifs that serve as docking sites for the endocytosis machinery and for cytoplasmic adaptor and scaffolding proteins involved in signaling events (Trommsdorff et al., J. Biol. Chem., 273:33556 (1998)).

LRP2, LRP4 and LRP8 are members of the low-density lipoprotein receptor (LDLR) family and mediate the endocytosis of an array of ligands. LRP2, also known as megalin or gp330, is a 600 kDa protein believed to play a role in neural development (McCarthy et al., J. Cell Sci., 116:955 (2003). LRP4 is a 1113 amino acid type II membrane-like protein that has been postulated to possess functions other than in lipid metabolism (Tomita et al., J. Biochem. (Tokyo) 124:784 (1998). LRP8, also known as ApoER2, is a 100 kDa protein that binds to the lipoprotein transporter apolipoprotein E (Ma et al., Neurosci. Lett. 332:216 (2002).

At present, the role LRP plays in disease is not understood. It is therefore desirable to clarify this role and design methods and compositions that are useful to address LRP associated disease.

### SUMMARY OF THE INVENTION

It is one of the objects of the present invention to provide LRP4, LRP8 and LRP2 (megalin) polypeptides; polynucleotides encoding such; and modulators of LRP4, LRP8 and LRP2 activity. The invention encompasses modulators that can, for example, bind to and interfere with the activity of at least one of LRP4, LRP8, LRP2 and active fragments thereof. The invention further provides for the use of pharmaceutical compositions containing such modulators for treatment of a disease, such as a proliferative disease or degenerative disease.

Modulators of the invention include nucleic acid molecules, small molecules, interfering RNA (RNAi) molecules, anti-sense molecules, and ribozymes.

Modulators include those that specifically interfere with the activity or binding of the polypeptides listed as SEQ ID NOS: 10-18 and 28-126.

The invention also employs modulators such as antibodies, including human or humanized antibodies. Antibodies of the invention may be polyclonal antibodies, monoclonal antibodies, single chain antibodies, agonist antibodies, antagonist antibodies, or neutralizing antibodies. Antibodies may also contain one or more of the following domains: a variable region of an immunoglobulin, a constant region of an immunoglobulin, a heavy chain of an immunoglobulin, a light chain of an immunoglobulin and an antigen-binding region of an immunoglobulin.

The invention also encompasses active fragments of antibodies as modulators of LRP4, LRP8 and LRP2 activity. An active fragment may be a fragment of an immunoglobulin that binds specifically to an antigen or an epitope. An active fragment may also be an Fc fragment, a cdr fragment, a V_{H} fragment, a V_{L} fragment, or a framework fragment.

Antibodies of the invention include those that specifically bind to or interfere with the activity of at least one polypeptide selected from LRP4, LRP8, LRP2 and active fragments thereof. Antibodies of the invention also include those that specifically bind to or interfere with the activity of a ligand of at least one polypeptide selected from LRP4, LRP8, LRP2 and active fragments thereof. The invention also provides antibodies that specifically bind to or interfere with the activity of a sequence selected from SEQ ID NOs: 10-18 and SEQ ID NOs: 28-126.

The invention provides pharmaceutical compositions and methods for administering such compositions to subjects to treat diseases, including proliferative diseases. Proliferative diseases treated by the invention include, for example, tumors and psoriasis. Many tumors may be treated by the invention, including splenic tumors, cervical tumors, leukemias, stomach tumors, liver tumors, thyroid tumors, skin tumors, breast tumors, lung tumors, kidney tumors, brain tumors, colon tumors, ovarian tumors, pancreatic tumors, and lymphomas.

The invention also provides pharmaceutical compositions and methods for treating degenerative diseases. Degenerative neural disease such as Alzheimer's disease may be so treated.

Compositions of the invention may be administered locally or systemically. Compositions may also be administered by intravenous, intraperitoneal, intratumor, intralesion, transdermal, intrathecal, subcutaneous, or intranasal administration or by inhalation.

The invention provides isolated polypeptide sequences as shown in SEQ ID NOs: 28-126. The invention also provides for nucleic acid molecules that encode such polypeptides. Vectors containing these nucleic acid sequences and a regulatory sequence that regulates the expression of the nucleic acid molecules are also provided.

The invention also encompasses modified cells containing the nucleic acids or vectors of the invention.

The antibodies of the invention also include isolated antibodies with an antigen-binding domain that binds to or interferes with the activity of the polypeptides of the invention. The invention further provides antibodies displayed on a bacteriophage.

The antibodies of the invention may also be associated with a therapeutic agent. For example, antibodies may be covalently linked to the therapeutic agent. Suitable therapeutic agents include toxins, radioactive isotopes, cytotoxic agents, and chemotherapeutic agents.

The invention also provides a method for detecting a polypeptide in a biological sample. For example, a biological sample may be contacted with an antibody of the invention and the presence of an antibody/polypeptide complex may be determined.

### BRIEF DESCRIPTION OF THE TABLES AND DRAWINGS

Table 1 shows the polypeptide sequences from SEQ.ID.NO:28 to SEQ.ID.NO:126, which represent pfam domain fragments from LRP4, LRP8, and LRP2. SEQ.ID.NO:1 to SEQ.ID.NO:27 are in the public domain and can be accessed through the National Center for Biotechnology Information database

In Table 2 (SEQ.ID.NO table), column 1 shows an internal designation ID number. Column 2 shows the nucleotide sequence ID number for the open reading frame (“ORF”) nucleotide sequence, column 3 shows the amino acid sequence ID number for the polypeptide sequence, column 4 shows the nucleotide sequence ID number for the entire nucleotide sequence, column 5 shows the classification/gene family, column 6 shows the polypeptide ID number of the source clone or sequence (source (aa)), and column 7 shows the nucleotide ID number of the source clone or sequence (source (nt)).

Table 3 shows the polypeptide alignment of all EGF domains in LRP4, LRP8, and LRP2.

Table 4 shows the polypeptide alignment of all Ldl_recept_a domains in LRP4, LRP8, and LRP2.

Table 5 shows the polypeptide alignment of all Ldl_recept_b domains in LRP4, LRP8, and LRP2.

Table 6 shows the polypeptide alignment of all EGF domains in LRP4.

Table 7 shows the polypeptide alignment of all Ldl_recept_b domains in LRP4.

Table 8 shows the polypeptide alignment of all EGF domains in LRP2.

Table 9 shows the polypeptide alignment of all Ldl_recep_a domains in LRP2.

Table 10 shows the polypeptide alignment of all Ldl_recept_b domains in LRP2.

Table 11 shows the polypeptide alignment of all Ldl_recept_a domains in LRP8.

Table 12 shows the polypeptide alignment of all Ldl_recept_b domains in LRP8

Table 13 shows the pfam domains and coordinates in LRP4. Column 1 shows the ID of the polypeptide, column 2 shows the name of the pfam domain, column 3 shows the start coordinate of the pfam domain in the polypeptide, and column 4 shows the stop coordinate of the pfam domain in the polypeptide.

Table 14 shows the pfam domains and coordinates in LRP2. Column 1 shows the ID of the polypeptide, column 2 shows the name of the pfam domain, column 3 shows the start coordinate of the pfam domain in the polypeptide, and column 4 shows the stop coordinate of the pfam domain in the polypeptide.

Table 15 shows the pfam domains and coordinates in LRP8. Column 1 shows the ID of the polypeptide, column 2 shows the name of the pfam domain, column 3 shows the start coordinate of the pfam domain in the polypeptide, and column 4 shows the stop coordinate of the pfam domain in the polypeptide.

The asterisks (*) indicate shared amino acid residues. The colons “:” indicate conserved amino acid changes. The hyphens “-” indicate amino acid residues that are missing from the novel sequence.

### DETAILED DESCRIPTION OF THE INVENTION

### Definitions

The terminologies used herein have their ordinary meanings. Further, the present invention can be more readily understood in light of the following particular definitions.

The terms “polypeptide,” “peptide,” and “protein,” used interchangeably herein, refer to a polymeric form of amino acids of any length, which can include naturally-occurring amino acids, coded and non-coded amino acids, chemically or biochemically modified, derivatized, or designer amino acids, amino acid analogs, peptidomimetics, and depsipeptides, and polypeptides having modified, cyclic, bicyclic, depsicyclic, or depsibicyclic peptide backbones. The term includes fragments of the Ig domains as described herein, single chain protein as well as multimers. The term also includes conjugated proteins, fusion proteins, including, but not limited to, GST fusion proteins, fusion proteins with a heterologous amino acid sequence, fusion proteins with heterologous and homologous leader sequences, fusion proteins with or without N-terminal methionine residues, pegalyated proteins, and immunologically tagged proteins. Also included in this term are variations of naturally occurring proteins, where such variations are homologous or substantially similar to the naturally occurring protein, as well as corresponding homologs from different species. Variants of polypeptide sequences include insertions, additions, deletions, or substitutions compared with the subject polypeptides. The term also includes peptide aptamers.

A “composition” of modulators, polypeptides, or polynucleotides herein refers to a composition that usually contains a pharmaceutically acceptable carrier or excipient that is conventional in the art and which is suitable for administration into a subject for therapeutic, diagnostic, or prophylactic purposes. For example, compositions for oral administration can include solutions, suspensions, tablets, pills, capsules, sustained release formulations, oral rinses, or powders.

The terms “agent and modulator” are used interchangeably herein. These terms refer to a substance that binds to or modulates a level or activity of a subject polypeptide or a level of mRNA encoding a subject protein or nucleic acid, or that modulates the activity of a cell containing the subject protein or nucleic acid. Where the agent modulates a level of mRNA encoding a subject protein, agents include ribozymes, antisense, and RNAi molecules. Where the agent is a substance that modulates a level of activity of a subject polypeptide, agents include antibodies specific for the subject polypeptide, peptide aptamers, small molecules, agents that bind a ligand-binding site in a subject polypeptide, and the like. Antibody agents include antibodies that specifically bind a subject polypeptide and activate the polypeptide, such as receptor-ligand binding that initiates signal transduction; antibodies that specifically bind a subject polypeptide and inhibit binding of another molecule to the polypeptide, thus preventing activation of a signal transduction pathway; antibodies that bind a subject polypeptide to modulate transcription; antibodies that bind a subject polypeptide to modulate translation; as well as antibodies that bind a subject polypeptide on the surface of a cell to initiate antibody-dependent cytotoxicity (“ADCC”) or to initiate cell killing or cell growth. Small molecule agents include those that bind the polypeptide to modulate activity of the polypeptide or cell containing the polypeptide in a similar fashion. The term “modulator” also refers to substances that modulate a condition or disorder associated with a subject polynucleotide or polypeptide. Such agents include subject polynucleotides themselves, subject polypeptides themselves, and the like. Agents may be chosen from amongst candidate agents, as defined below.

An “active fragment” is a fragment having structural, regulatory, or biochemical functions of a naturally occurring molecule or any function related to or associated with a metabolic or physiological process. For example, a fragment demonstrates activity when it participates in a molecular interaction with another molecule, when it has therapeutic value in alleviating a disease condition, or when it has prophylactic value in preventing or reducing the occurrence of disease, or when it induces an immune response to the molecule. Active polypeptide fragments include those exhibiting activity similar, but not necessarily identical, to an activity of a polypeptide set forth herein. The activity may include an improved desired activity, or a decreased undesired activity.

The term “anti-sense molecule” includes antisense oligonucleotides (ODN), i.e., synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g., by reducing the amount of mRNA available for translation, through activation of RNase H, or steric hindrance. One or a combination of antisense molecules can be administered, where a combination can comprise multiple different sequences. Antisense molecules can be produced by expression of all or a part of the target gene sequence in an appropriate vector, where the transcriptional initiation is oriented such that an antisense strand is produced as an RNA molecule. Alternatively, the antisense molecule is a synthetic oligonucleotide.

An “interfering RNA (RNAi)” molecule is an RNA molecule that partially or completely silences one or more eukaryotic genes. For example, double stranded RNA can induce the homology-dependent degradation of its cognate mRNA. Use of RNAi to reduce a level of a particular mRNA and/or protein is based on the interfering properties of double-stranded RNA derived from the coding regions of a gene. The technique can reduce the time between identifying an interesting gene sequence and understanding its function, and thus is an efficient high-throughput method for disrupting gene function. RNAi can also help identify the biochemical mode of action of a drug and identify other genes encoding products that can respond or interact with specific compounds.

A “biologically active” entity, or an entity having “biological activity,” is one having structural, regulatory, or biochemical functions of a naturally occurring molecule or any function related to or associated with a metabolic or physiological process. Biologically active polynucleotide fragments are those exhibiting activity similar, but not necessarily identical, to an activity of a polynucleotide of the present invention. The biological activity can include an improved desired activity, or a decreased undesirable activity. For example, an entity demonstrates biological activity when it participates in a molecular interaction with another molecule, or when it has therapeutic value in alleviating a disease condition, or when it has prophylactic value in inducing an immune response to the molecule, or when it has diagnostic value in determining the presence of the molecule, such as a biologically active fragment of a polynucleotide that can be detected as unique for the polynucleotide molecule, or that can be used as a primer in PCR.

The terms “polynucleotide,” “nucleotide,” “nucleic acid,” “polynucleic molecule,” “nucleotide molecule,” “nucleic acid molecule,” “nucleic acid sequence,” “polynucleotide sequence,” and “nucleotide sequence” are used interchangeably herein to refer to polymeric forms of nucleotides of any length. The polynucleotides can contain deoxyribonucleotides, ribonucleotides, and/or their analogs or derivatives. For example, nucleic acids can be naturally occurring DNA or RNA, or can be synthetic analogs, as known in the art. The terms also encompass genomic DNA, genes, gene fragments, exons, introns, regulatory sequences or regulatory elements (such as promoters, enhancers, initiation and termination regions, other control regions, expression regulatory factors, and expression controls), DNA comprising one or more single-nucleotide polymorphisms (SNPs), allelic variants, isolated DNA of any sequence, and cDNA. The terms also encompass mRNA, TRNA, rRNA, ribozymes, splice variants, antisense RNA, antisense conjugates, RNAi, and isolated RNA of any sequence. The terms also encompass recombinant polynucleotides, heterologous polynucleotides, branched polynucleotides, labeled polynucleotides, hybrid DNA/RNA, polynucleotide constructs, vectors comprising the subject nucleic acids, nucleic acid probes, primers, and primer pairs. The polynucleotides can comprise modified nucleic acid molecules, with alterations in the backbone, sugars, or heterocyclic bases, such as methylated nucleic acid molecules, peptide nucleic acids, and nucleic acid molecule analogs, which may be suitable as, for example, probes if they demonstrate superior stability and/or binding affinity under assay conditions. Analogs of purines and pyrimidines, including radiolabeled and fluorescent analogs, are known in the art. The polynucleotides can have any three-dimensional structure, and can perform any function, known or as yet unknown. The terms also encompass single-stranded, double-stranded and triple helical molecules that are either DNA, RNA, or hybrid DNA/RNA and that may encode a full-length gene or a biologically active fragment thereof. Biologically active fragments of polynucleotides can encode the polypeptides herein, as well as anti-sense and RNAi molecules. Thus, the full-length polynucleotides herein may be treated with enzymes, such as Dicer, to generate a library of short RNAi fragments that are within the scope of the present invention.

A “vector” is a plasmid that can be used to transfer DNA sequences from one organism to another. An “expression vector” is a cloning vector that contains regulatory sequences that allow transcription and translation of a cloned gene or genes and thus transcribe and clone DNA. Expression vectors can be used to express the polypeptides of the invention and typically include restriction sites to provide for the insertion of nucleic acid sequences encoding heterologous protein or RNA molecules. Artificially constructed “plasmids,” i.e., small, independently replicating pieces of extrachromosomal cytoplasmic DNA that can be transferred from one organism to another, are commonly used as cloning vectors.

A “bacteriophage” is a virus with a specific affinity for one or more type of bacteria, and which infect these bacteria. Bacteriophages generally comprise a capsid or protein coat that encloses the genetic material, i.e., the DNA or RNA that enters the bacterium when a bacteriophage infects a bacterium.

“Expression” of a nucleic acid molecule refers to the conversion of the information into a gene product. A gene product can be the direct transcriptional product of a gene (e.g., mRNA, tRNA, rRNA, antisense RNA, ribozyme, structural RNA, or any other type of RNA) or a protein produced by translation of an mRNA. Gene products also include RNAs that are modified, e.g., by processes such as capping, polyadenylation, methylation, and editing, and proteins modified by, for example, methylation, acetylation, phosphorylation, ubiquitination, ADP-ribosylation, myristilation, and glycosylation.

An “expression system” is any composition that permits protein synthesis when an expression vector is provided to the system. Expression systems are well-known by those skilled in the art. They include cell-free expression systems, e.g., wheat germ extract systems, rabbit reticulocyte systems, and frog oocyte systems. They also include systems that utilize host cells, such as E. coli expression systems, yeast expression systems, insect expression systems, insect expression systems, and mammalian expression systems.

A “regulatory sequence” of a polynucleotide is a sequence that aids the expression, including transcription and translation, of a coding sequence to which it is linked. The term includes, e.g., promoters, transcription termination sequences, upstream regulatory domains, polyadenylation signals, leader sequences, and enhancers.

To “regulate” is to govern or direct; it also refers to the process of providing order, method, or uniformity. “Regulation” is encompassed within modulation, and further encompasses the process of fixing or adjusting a time, amount, degree, or rate.

An “isolated,” “purified,” or “substantially isolated” polynucleotide, or a polynucleotide in “substantially pure form,” in “substantially purified form,” in “substantial purity,” or as an “isolate,” is one that is substantially free of the sequences with which it is associated in nature, or other nucleic acid sequences that do not include a sequence or fragment of the subject polynucleotides. By substantially free is meant that less than about 90%, less than about 80%, less than about 70%, less than about 60%, or less than about 50% of the composition is made up of materials other than the isolated polynucleotide. Where at least about 99% of the total macromolecules is the isolated polynucleotide, the polynucleotide is at least about 99% pure, and the composition comprises less than about 1% contaminant. Such isolated polynucleotides may be recombinant polynucleotides, modified, degenerate and homologous polynucleotides, and chemically synthesized polynucleotides, which, by virtue of origin or manipulation, are not associated with all or a portion of a polynucleotide with which it is associated in nature, are linked to a polynucleotide other than that to which it is linked in nature, or do not occur in nature. For example, the subject polynucleotides are generally provided as other than on an intact chromosome, and recombinant embodiments are typically flanked by one or more nucleotides not normally associated with the subject polynucleotide on a naturally-occurring chromosome.

A “gene,” for the purposes of the present disclosure, includes a DNA region encoding a gene product, as well as all DNA regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

The term “antibody” refers to protein generated by the immune system that is capable of recognizing and binding to a specific antigen. Antibodies, and methods of making antibodies, are commonly known in the art.

As used herein, the term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, e.g., Winter et al., Nature 349:293 (1991) and U.S. Pat. No. 4,816,567); F(ab′)₂ and F(ab) fragments; Fv molecules (noncovalent heterodimers (see, e.g., Inbar et al., Proc Natl Acad Sci USA 69:2659 (1972) and Ehrlich et al., Biochem 19:4091 (1980)); single-chain Fv molecules (sFv) (see, e.g., Huston et al., Proc Natl Acad Sci USA 85:5879 (1980)); dimeric and trimeric antibody fragment constructs; minibodies (see, e.g., Pack et al., Biochem 31:1579 (1992) and Cumber et al., J. Immunology 149B:120 (1992)); humanized antibody molecules (see, e.g., Riechmann et al., Nature 332:323 (1988) and Verhoeyan et al., Science 239:1534 (1988)); and, any functional fragments obtained from such molecules, wherein such fragments retain specific-binding.

An “antigen” is a substance that provokes an immune response.

An “epitope” is the site of an antigenic molecule to which an antibody binds.

An “agonist antibody” is one that mimics, enhances, stimulates, or activates the function of a molecule with which the agonist interacts.

An “antagonist antibody” is one that competes, inhibits, or interferes with the activity of a molecule with which the antagonist interacts. For example, an antagonist antibody may bind to the receptor without inducing an active response.

An “antigen-binding fragment (Fab fragment)” is a disulfide-linked heterodimer, each chain of which contains one immunoglobulin constant region (C) domain and one variable region (V) domain; the juxtaposition of the V domains forms the antigen-binding site. The two Fab fragments of an intact immunoglobulin molecule correspond to its two arms, which typically contain light chain regions paired with the V and C1 domains of the heavy chains.

A “Fragment crystallizable fragment (Fc fragment)” is the portion of an antibody molecule that interacts with effector molecules and cells. It comprises the carboxy-terminal portions of the immunoglobulin heavy chains. The functional differences between heavy-chain isotypes lie mainly in the Fc fragment.

The “constant region” of an antibody is its effector region, and determines the functional class of the antibody. The constant region of a heavy or light chain is located at or near the carboxyl terminus.

The “variable region” of an antibody is the region that binds to the antigen; it provides antibody specificity. The variable region of a heavy or light chain is located at or near the amino terminus. A “V_{H}” fragment contains the variable region of a heavy chain; a “V_{L}” fragment contains the variable region of a light chain.

An “immunoglobulin” is an antibody molecule, i.e., a polypeptide that can respond to a foreign molecule of invading organism, e.g., by binding to it, marking it for destruction, and/or inactivating it.

A “heavy chain” is the larger of the two classes of polypeptide chains that combine to form immunoglobulin molecules. The class of the heavy chain determines the class of the immunoglobulin, e.g., IgG, IgA, IgE, IgD, or IgM.

A “light chain” is the smaller of the two classes of polypeptide chains that combine to form immunoglobulin molecules. Light chains are generally classified into two classes, kappa and lambda, on the basis of structural differences in their constant regions.

The “complementarity-determining region (cdr)” is the three dimensional structure of an antibody that provides antigenic specificity.

A “framework fragment” is that region of the variable domain that contains relatively invariant sequences and lies between the hypervariable regions. Framework regions provide a protein scaffold for the hypervariable regions.

A “humanized” antibody is an antibody that contains mostly human immunoglobulin sequences. This term is generally used to refer to a non-human immunoglobulin that has been modified to incorporate portions of human sequences, and may include a human antibody that contains entirely human immunoglobulin sequences.

A “single chain antibody” is an Fab fragment comprising only the V domain of a heavy chain linked by a peptide to a V domain of a light chain.

A “polyclonal antibody” a mixture of antibodies of different specificities, as in the serum of an animal immunized to various antigens or epitopes.

A “monoclonal antibody” is an antibody composition having a homogeneous antibody population. The term is not limited with regard to the species or source of the antibody, nor by the manner in which it is made. The term encompasses whole immunoglobulins and immunoglobulin fragments.

Methods of making polyclonal and monoclonal antibodies are known in the art. Polyclonal antibodies are generated by immunizing a suitable animal, such as a mouse, rat, rabbit, sheep or goat, with an antigen of interest, such as a stem cell transformed with a gene encoding an antigen. In order to enhance immunogenicity, the antigen can be linked to a carrier prior to immunization. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the antigen may be conjugated to a bacterial toxoid, such as toxoid from diphtheria, tetanus, cholera, etc., in order to enhance the immunogenicity thereof.

The term “binds specifically,” in the context of antibody binding, refers to high avidity and/or high affinity binding of an antibody to a specific polypeptide, or more accurately, to an epitope of a specific polypeptide. Antibody binding to such epitope on a polypeptide can be stronger than binding of the same antibody to any other epitopes, particularly other epitopes that can be present in molecules in association with, or in the same sample as the polypeptide of interest. For example, when an antibody binds more strongly to one epitope than to another, adjusting the binding conditions can result in antibody binding almost exclusively to the specific epitope and not to any other epitopes on the same polypeptide, and not to any other polypeptide, which does not comprise the epitope. Antibodies that bind specifically to a subject polypeptide may be capable of binding other polypeptides at a weak, yet detectable, level (e.g., 10% or less of the binding shown to the polypeptide of interest). Such weak binding, or background binding, is readily discernible from the specific antibody binding to a subject polypeptide, e.g., by use of appropriate controls. In general, antibodies of the invention bind to a specific polypeptide with a binding affinity of 10⁻⁷ M or greater (e.g., 10⁻⁸ M, 10⁻⁹ M, 10⁻¹⁰ M, 10⁻¹¹ M, etc.).

A “cytotoxic agent” includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrolestosterone, glucocorticoids, procaine, tetracaine, lidocaine, propranolol, and puromycin and analogs or homologs thereof. A “therapeutic agent” is any agent that may be administered to a subject as part of a therapeutic regimen or treatment for a disease or condition. Therapeutic agents may or may not be cytotoxic. Therapeutic agents include, but are not limited to, toxins, radioactive isotopes, cytotoxic agents, chemotherapeutic agents, and drug moieties. Examples include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The terms “linked,” “link,” “linkage,” and “linking,” may be used interchangeably herein, and refer to covalent and non-covalent conjugation between a molecule and an agent. For example, in certain embodiments of the invention, a molecule, such as an antibody, can be conjugated to an agent, such as a cytotoxic compound. Methods of preparation of linkers and cytotoxic compounds are know in the art, and can be found, for example, in PCT publication WO 03/061694. Cytotoxic compounds of the invention include, for example, capecitabine, carboplatin, chlorambuoll, cisplatin, cyclophospamide, cytarabine, dacarbazine, daunorubicin, docetaxel, doxorubicin, epirubicin, estrmustine, etoposide phosphate, fludarabine, fluorouracil, 5-FU, gemcitabine, hydroxyurea, idarubicin, ifosfamide, irinotecan, eucovorin, meclorethamine nitrogen mustard, methotrexate, mitomycin C, mitoxantrone, oxalilplatin, paclitaxel, tamoxifen, topotecan, vinblastine, vincristine, and vinorelbine.

A “disease” is a pathological, abnormal, and/or harmful condition of an organism. The term includes conditions, syndromes, and disorders. A “proliferative disease” is a disease or disorder that involves abnormal cell proliferation, including, but not limited to, cancer, psoriasis, and scleroderma.

“Cancer” is herein defined as any abnormal malignant cell or tissue growth, e.g., a tumor. It is characterized by the proliferation of abnormal cells that tend to invade the surrounding tissue and metastasize to new body sites. Cancer encompasses carcinomas, which are cancers of epithelial cells, and are the most common forms of human cancer; carcinomas include squamous cell carcinoma, adenocarcinoma, melanomas, and hepatomas. Cancer also encompasses sarcomas, which are tumors of mesenchymal origin, and includes osteogenic sarcomas, leukemias, and lymphomas. Cancers also encompasses leukemias and lymphomas, and can have one or more than one neoplastic cell type.

A “tumor” is a solid mass of cells or tissues undergoing uncontrolled proliferative growth. Uncontrolled proliferative growth is any abnormal cell or tissue growth that does not respond to one or more inhibitory signals. It may be benign or malignant. Uncontrolled proliferative cell growth includes cancer.

“Treatment,” “treating,” and the like, as used herein, refer to obtaining a desired pharmacologic and/or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse affect attributable to the disorder. That is, “treatment” includes (1) preventing the disorder from occurring or recurring in a subject who may be predisposed to the disorder but has not yet been diagnosed as having it, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as inflammation, pain, and/or tumor size.

By “fragment” is intended a polypeptide, e.g., Ig domains, consisting of only a part of the intact full-length polypeptide sequence and structure. The fragment can include a C-terminal deletion an N-terminal deletion, and/or an internal deletion of the native polypeptide. A fragment of a protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence.

As noted above, a “biologically active” entity, or an entity having “biological activity,” is one having structural, regulatory, or biochemical functions of a naturally occurring molecule or any function related to or associated with a metabolic or physiological process. Biologically active polypeptide fragments are those exhibiting activity similar, but not necessarily identical, to an activity of a polypeptide of the present invention. The biological activity can include an improved desired activity, or a decreased undesirable activity. For example, an entity demonstrates biological activity when it participates in a molecular interaction with another molecule, or when it has therapeutic value in alleviating a disease condition, or when it has prophylactic value in inducing an immune response to the molecule, or when it has diagnostic value in determining the presence of the molecule. A biologically active polypeptide or fragment thereof includes one that can participate in a biological reaction, for example, as a transcription factor that combines with other transcription factors for initiation of transcription, or that can serve as an epitope or immunogen to stimulate an immune response, such as production of antibodies, or that can transport molecules into or out of cells, or that can perform a catalytic activity, for example polymerization or nuclease activity, or that can participate in signal transduction by binding to receptors, proteins, or nucleic acids, activating enzymes or substrates.

An “isolated,” “purified,” or “substantially isolated” polypeptide, or a polypeptide in “substantially pure form,” in “substantially purified form,” in “substantial purity,” or as an “isolate,” is one that is substantially free of the materials with which it is associated in nature or other polypeptide sequences that do not include a sequence or fragment of the subject polypeptides. By substantially free is meant that less than about 90%, less than about 80%, less than about 70%, less than about 60%, or less than about 50% of the composition is made up of materials other than the isolated polypeptide. Where at least about 99% of the total macromolecules is the isolated polypeptide, the polypeptide is at least about 99% pure, and the composition comprises less than about 1% contaminant. Such isolated polypeptides may be recombinant polypeptides, modified, tagged and fusion polypeptides, and chemically synthesized polypeptides, which by virtue or origin or manipulation, are not associated with all or a portion of the materials with which they are associated in nature, are linked to molecules other than that to which they are linked in nature, or do not occur in nature.

Detection methods of the invention can be qualitative or quantitative. Thus, as used herein, the terms “detecting,” “identifying,” “determining,” and the like, refer to both qualitative and quantitative determinations, and include “measuring.” For example, detection methods include methods for detecting the presence and/or level of polynucleotide or polypeptide in a biological sample, and methods for detecting the presence and/or level of biological activity of polynucleotide or polypeptide in a sample.

“Biological sample,” as used herein, includes biological fluids such as blood, serum, plasma, urine, cerebrospinal fluid, tears, saliva, lymph, dialysis fluid, lavage fluid, semen, and other liquid samples or tissues of biological origin. It includes cells or cells derived therefrom and the progeny thereof, including cells in culture, cell supernatants, and cell lysates. It includes organ or tissue culture derived fluids, tissue biopsy samples, tumor biopsy samples, stool samples, and fluids extracted from physiological tissues. Cells dissociated from solid tissues, tissue sections, and cell lysates are included. The definition also includes samples that have been manipulated in any way after their procurement, such as by treatment with reagents, solubilization, or enrichment for certain components, such as polynucleotides or polypeptides. Also included in the term are derivatives and fractions of biological samples. A biological sample can be used in a diagnostic or monitoring assay.

The term “modified cell” includes an individual cell, cell line, cell culture, or in vivo cell, which has been a recipient of any polynucleotides or polypeptides of the invention, for example, a recombinant vector, an isolated polynucleotide, antibody or fusion protein. Modified cells include progeny of a single modified cell, and the progeny may not necessarily be completely identical (in morphology, physiology, or in total DNA, RNA, or polypeptide complement) to the original parent cell due to natural, accidental, or deliberate mutation and/or change. Modified cells can be prokaryotic or eukaryotic, including mammalian, insect, amphibian, reptile, crustacean, avian, fish, plant and fungal cells. A modified cell includes cells transformed, transfected, transduced, or infected in vivo or in vitro with a polynucleotide of the invention, for example, a recombinant vector. A host modified that comprises a recombinant vector of the invention may be called a “recombinant modified cell.”

The terms “individual,” “host,” “patient,” and “subject,” used interchangeably herein, refer to a mammal, including, but not limited to, murines, simians, humans, felines, canines, equines, bovines, porcines, ovines, caprines, mammalian farm animals, mammalian sport animals, and mammalian pets. “Mammals” or “mammalian,” are used broadly to describe organisms which are within the class mammalia, including the orders carnivore (e.g., dogs and cats), rodentia (e.g., mice, guinea pigs, and rats), and other mammals, including cattle, goats, sheep, cows, horses, rabbits, and pigs, and primates (e.g., humans, chimpanzees, and monkeys).

The terms “candidate agent,” “subject agent,” or “test agent,” used interchangeably herein, encompass numerous chemical classes, typically synthetic, semi-synthetic, or naturally occurring inorganic or organic molecules, small molecules, or macromolecular complexes. Candidate agents can be small organic compounds having a molecular weight of more than about 50 and less than about 2,500 daltons. Candidate agents can comprise functional groups necessary for structural interaction with proteins, particularly hydrogen bonding, and can include at least an amine, carbonyl, hydroxyl or carboxyl group, and can contain at least two of the functional chemical groups. The candidate agents can comprise cyclical carbon or heterocyclic structures and/or aromatic or polyaromatic structures substituted with one or more of the above functional groups. Candidate agents are also found among biomolecules, including oligonucleotides, polynucleotides, and fragments thereof, depsipeptides, polypeptides and fragments thereof, oligosaccharides, polysaccharides and fragments thereof, lipids, fatty acids, steroids, purines, pyrimidines, derivatives thereof, structural analogs, modified nucleic acids, modified, derivatized or designer amino acids, or combinations thereof.

An “agent which modulates a biological activity of a subject polypeptide,” as used herein, describes any substance, synthetic, semi-synthetic, or natural, organic or inorganic, small molecule or macromolecular, pharmaceutical or protein, with the capability of altering a biological activity of a subject polypeptide or of a fragment thereof, as described herein. Generally, a plurality of assay mixtures is run in parallel with different agent concentrations to obtain a differential response to the various concentrations. Typically, one of these concentrations serves as a negative control, i.e., at zero concentration or below the level of detection. The biological activity can be measured using any assay known in the art.

An agent which modulates a biological activity of a subject polypeptide increases or decreases the activity at least about 10%, at least about 15%, at least about 20%, at least about 25%, at least about 50%, at least about 100%, or at least about 2-fold, at least about 5-fold, or at least about 10-fold or more when compared to a suitable control.

The term “agonist” refers to a substance that mimics the function of an active molecule. Agonists include, but are not limited to, drugs, hormones, antibodies, and neurotransmitters, as well as analogues and fragments thereof.

The term “antagonist” refers to a molecule that competes for the binding sites of an agonist, but does not induce an active response. Antagonists include, but are not limited to, drugs, hormones, antibodies, and neurotransmitters, as well as analogues and fragments thereof.

The term “ligand” refers to any molecule that binds to a specific site on another molecule.

The term “modulate” encompasses an increase or a decrease, a stimulation, inhibition, or blockage in the measured activity when compared to a suitable control. “Modulation” of expression levels includes increasing the level and decreasing the level of an mRNA or polypeptide encoded by a polynucleotide of the invention when compared to a control lacking the agent being tested. In some embodiments, agents of particular interest are those which inhibit a biological activity of a subject polypeptide, and/or which reduce a level of a subject polypeptide in a cell, and/or which reduce a level of a subject mRNA in a cell and/or which reduce the release of a subject polypeptide from a eukaryotic cell. In other embodiments, agents of interest are those that increase a biological activity of a subject polypeptide, and/or which increase a level of a subject polypeptide in a cell, and/or which increase a level of a subject mRNA in a cell and/or which increase the release of a subject polypeptide from a eukaryotic cell.

An agent that “modulates the level of expression of a nucleic acid” in a cell is one that brings about an increase or decrease of at least about 1.25-fold, at least about 1.5-fold, at least about 2-fold, at least about 5-fold, at least about 10-fold, or more in the level (i.e., an amount) of mRNA and/or polypeptide following cell contact with a candidate agent compared to a control lacking the agent.

A “pharmaceutically acceptable carrier,” “pharmaceutically acceptable diluent,” or “pharmaceutically acceptable excipient,” or “pharmaceutically acceptable vehicle,” used interchangeably herein, refer to a non-toxic solid, semisolid or liquid filler, diluent, encapsulating material or formulation auxiliary of any conventional type. A pharmaceutically acceptable carrier is non-toxic to recipients at the dosages and concentrations employed and is compatible with other ingredients of the formulation. For example, the carrier for a formulation containing polypeptides would not normally include oxidizing agents and other compounds that are known to be deleterious to polypeptides. Suitable carriers include, but are not limited to, water, dextrose, glycerol, saline, ethanol, and combinations thereof. The carrier can contain additional agents such as wetting or emulsifying agents, pH buffering agents, or adjuvants which enhance the effectiveness of the formulation. Adjuvants of the invention include, but are not limited to Freunds's, Montanide ISA Adjuvants [Seppic, Paris, France], Ribi's Adjuvants (Ribi ImmunoChem Research, Inc., Hamilton, Mont.), Hunter's TiterMax (CytRx Corp., Norcross, Ga.), Aluminum Salt Adjuvants (Alhydrogel—Superfos of Denmark/Accurate Chemical and Scientific Co., Westbury, N.Y.), Nitrocellulose-Adsorbed Protein, Encapsulated Antigens, and Gerbu Adjuvant (Gerbu Biotechnik GmbH, Gaiberg, Germany/C—C Biotech, Poway, Calif.). Topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents can be added as necessary. Percutaneous penetration enhancers such as Azone can also be included.

“Pharmaceutically acceptable salts” include the acid addition salts (formed with the free amino groups of the polypeptide) and which are formed with inorganic acids such as, for example, hydrochloric or phosphoric acids, or such organic acids as acetic, mandelic, oxalic, and tartaric. Salts formed with the free carboxyl groups can also be derived from inorganic bases such as, for example, sodium, potassium, ammonium, calcium, or ferric hydroxides, and such organic bases as isopropylamine, trimethylamine, 2-ethylamino ethanol, and histidine.

Compositions for oral administration can form solutions, suspensions, tablets, pills, capsules, sustained release formulations, oral rinses, or powders.

The term “unit dosage form,” as used herein, refers to physically discrete units suitable as unitary dosages for human and animal subjects, each unit containing a predetermined quantity of compounds of the present invention calculated in an “effective amount,” that is, a dosage sufficient to produce the desired result or effect in association with a pharmaceutically acceptable carrier. The specifications for the novel unit dosage forms of the present invention depend on the particular compound employed, the host, and the effect to be achieved, as well as the pharmacodynamics associated with each compound in the host.

### Screening and Diagnostic Methods

1. Identifying Biological Molecules that Interact with a Polypeptide

Formation of a binding complex between a LRP2, LRP4 or LRP8 polypeptide and an interacting polypeptide or other macromolecule (e.g., DNA, RNA, lipids, polysaccharides, and the like) can be detected using any known method. Suitable methods include: a yeast two-hybrid system (Zhu et al., Proc. Natl. Acad. Sci. USA 94:13,063 (1997); Fields et al., Nature 340:245 (1989); U.S. Pat. No. 5,283,173; Chien et al., Proc. Natl. Acad. Sci. USA 88:9578 (1991); a mammalian cell two-hybrid method; a fluorescence resonance energy transfer (FRET) assay; a bioluminescence resonance energy transfer (BRET) assay; a fluorescence quenching assay; a fluorescence anisotropy assay (Jameson et al., Methods Enzymol. 246:283 (1995); an immunological assay; and an assay involving binding of a detectably labeled protein to an immobilized protein.

2. Detecting mRNA Levels and Monitoring Gene Expression

The present invention provides methods for detecting the presence of a LRP2, LRP4 or LRP8 mRNA in a biological sample. The methods can be used, for example, to assess whether a test compound affects gene expression, either directly or indirectly. The present invention provides diagnostic methods to compare the abundance of a nucleic acid with that of a control value, either qualitatively or quantitatively, and to relate the value to a normal or abnormal expression pattern.

Methods of measuring mRNA levels are known in the art, as described in for example, WO 97/27317. These methods generally comprise contacting a sample with a polynucleotide of the invention under conditions that allow hybridization and detecting hybridization, if any, as an indication of the presence of the polynucleotide of interest. Detection can be accomplished by any known method, including, but not limited to, in situ hybridization, PCR, RT-PCR, and “Northern” or RNA blotting, or combinations of such techniques, using a suitably labeled subject polynucleotideA common method employed is use of microarrays which can be purchased or customized, for example, through conventional vendors such as Affymetrix.

3. Detecting and Monitoring Polypeptide Presence and Biological Activity

The present invention provides methods for detecting the presence and/or biological activity of a LRP2, LRP4 or LRP8 polypeptide in a biological sample. The assay used will be appropriate to the biological activity of the particular polypeptide. Thus, e.g., where the biological activity is binding to a second macromolecule, the assay detects protein-protein binding, protein-DNA binding, protein-carbohydrate binding, or protein-lipid binding, as appropriate, using well known assays. Where the biological activity is signal transduction (e.g., transmission of a signal from outside the cell to inside the cell) or transport, an appropriate assay is used, such as measurement of intracellular calcium ion concentration, measurement of membrane conductance changes, or measurement of intracellular potassium ion concentration.

The present invention also provides methods for detecting the presence or measuring the level of a normal or abnormal LRP2, LRP4 or LRP8 polypeptide in a biological sample using a specific antibody. The methods generally comprise contacting the sample with a specific antibody and detecting binding between the antibody and molecules of the sample. Specific antibody binding, when compared to a suitable control, is an indication that a polypeptide of interest is present in the sample.

A variety of methods to detect specific antibody-antigen interactions are known in the art, e.g., standard immunohistological methods, immunoprecipitation, enzyme immunoassay, and radioimmunoassay. Briefly, antibodies are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, specific-binding pairs may be used, involving, e.g., a second stage antibody or reagent that is detectably-labeled, as described above. Such reagents and their methods of use are well known in the art

4. Modulating mRNA and Peptides in Biological Samples

The present invention provides screening methods for identifying agents that modulate the level of a LRP2, LRP4 or LRP8 mRNA molecule of the invention, agents that modulate the level of a polypeptide of the invention, and agents that modulate the biological activity of a polypeptide of the invention. In some embodiments, the assay is cell-free; in others, it is cell-based. Where the screening assay is a binding assay, one or more of the molecules can be joined to a label, where the label can directly or indirectly provide a detectable signal.

In these embodiments, the candidate agent is combined with a cell possessing a polynucleotide transcriptional regulatory element operably linked to a polypeptide-coding sequence of interest, e.g., a subject cDNA or its genomic component; and determining the agent's effect on polynucleotide expression, as measured, for example by the level of mRNA, polypeptide, or fusion polypeptide

In other embodiments, for example, a recombinant vector can comprise an isolated polynucleotide transcriptional regulatory sequence, such as a promoter sequence, operably linked to a reporter gene (e.g., β-galactosidase, CAT, luciferase, or other gene that can be easily assayed for expression). In these embodiments, the method for identifying an agent that modulates a level of expression of a polynucleotide in a cell comprises combining a candidate agent with a cell comprising a transcriptional regulatory element operably linked to a reporter gene; and determining the effect of said agent on reporter gene expression.

Known methods of measuring mRNA levels can be used to identify agents that modulate LRP2, LRP4 or LRP8 mRNA levels, including, but not limited to, PCR with detectably-labeled primers. Similarly, agents that modulate polypeptide levels can be identified using standard methods for determining polypeptide levels, including, but not limited to an immunoassay such as ELISA with detectably-labeled antibodies.

A wide variety of cell-based assays can also be used to identify agents that modulate eukaryotic or prokaryotic mRNA and/or polypeptide levels. Examples include transformed cells that over-express a cDNA construct and cells transformed with a polynucleotide of interest associated with an endogenously-associated promoter operably linked to a reporter gene. Expression levels are measured and compared in the test and control samples.

The present invention further provides methods of identifying agents that modulate a biological activity of LRP2, LRP4 or LRP8 polypeptides. The method generally comprises contacting a test agent with a sample containing the subject polypeptide and assaying a biological activity of the subject polypeptide in the presence of the test agent. An increase or a decrease in the assayed biological activity in comparison to the activity in a suitable control (e.g., a sample comprising a subject polypeptide in the absence of the test agent) is an indication that the substance modulates a biological activity of the subject polypeptide. The mixture of components is added in any order that provides for the requisite interaction.

Accordingly, the present invention provides a method for identifying an agent, particularly a biologically active agent that modulates the level of expression of a LRP2, LRP4 or LRP8 nucleic acid in a cell, the method comprising: combining a candidate agent to be tested with a cell comprising a nucleic acid that encodes the polypeptide, and determining the agent's effect on polypeptide expression.

Agents that decrease a LRP2, LRP4 or LRP8 biological activity can find use in treating disorders associated with the biological activity of the molecule. Alternatively, some embodiments will detect agents that increase a biological activity. Agents that increase a biological activity of a molecule of the invention can find use in treating disorders associated with a deficiency in the biological activity.

A variety of different candidate agents can be screened by the above methods. Candidate agents encompass numerous chemical classes, as described above.

Candidate agents are obtained from a wide variety of sources including libraries of synthetic or natural compounds. Numerous means are available for random and directed synthesis of a wide variety of organic compounds and biomolecules, including expression of randomized oligonucleotides and oligopeptides. For example, random peptide libraries obtained by yeast two-hybrid screens (Xu et al., Proc. Natl. Acad. Sci. USA 94:12473 (1997), phage libraries (Hoogenboom et al., Immunotechnology 4:1 (1998), or chemically generated libraries. Alternatively, libraries of natural compounds in the form of bacterial, fungal, plant and animal extracts are available or readily produced, including antibodies produced upon immunization of an animal with subject polypeptides, or fragments thereof, or with the encoding polynucleotides. Additionally, natural or synthetically produced libraries and compounds are readily modified through conventional chemical, physical and biochemical means, and can be used to produce combinatorial libraries. Further, known pharmacological agents can be subjected to directed or random chemical modifications, such as acylation, alkylation, esterification, and amidification, etc, to produce structural analogs.

5. Kits

The present invention provides methods for diagnosing disease states based on the detected presence and/or level of a LRP2, LRP4 or LRP8 polynucleotide or polypeptide in a biological sample, and/or the detected presence and/or level of biological activity of a LRP2, LRP4 or LRP8 polynucleotide or polypeptide. These detection methods can be provided as part of a kit. Thus, the invention further provides kits for detecting the presence and/or a level of a polynucleotide or polypeptide in a biological sample and/or or the detected presence and/or level of biological activity of the polynucleotide or polypeptide.

Where the kit provides for LRP2, LRP4 or LRP8 polypeptide detection, it can include one or more specific antibodies. In some embodiments, the antibody specific to the polypeptide is detectably labeled. In other embodiments, the antibody specific to the polypeptide is not labeled; instead, a second, detectably-labeled antibody is provided that binds to the specific antibody. The kit may further include blocking reagents, buffers, and reagents for developing and/or detecting the detectable marker. The kit may further include instructions for use, controls, and interpretive information.

The present invention provides for kits with unit doses of an active agent. In some embodiments, the agent is provided in oral or injectable doses. Such kits will comprise containers containing the unit doses and an informational package insert describing the use and attendant benefits of the drugs in treating a condition of interest.

### Therapeutic Compositions

The invention further provides agents identified using a screening assay of the invention, and compositions comprising the agents, subject polypeptides, subject polynucleotides, modulators thereof including antibodies, recombinant vectors, and/or host cells, including pharmaceutical compositions containing such in a pharmaceutically acceptable carrier or excipient for therapeutic administration. The subject compositions can be formulated using well-known reagents and methods. These compositions can include a buffer, which is selected according to the desired use of the agent, polypeptide, polynucleotide, recombinant vector, or host cell, and can also include other substances appropriate to the intended use. Those skilled in the art can readily select an appropriate buffer, a wide variety of which are known in the art, suitable for an intended use.

1. Excipients and Formulations

In some embodiments, compositions are provided in formulation with pharmaceutically acceptable excipients, a wide variety of which are known in the art (Gennaro, Remington: The Science and Practice of Pharmacy, 20th ed., Lippincott, Williams, & Wilkins (2000); Ansel et al., Pharmaceutical Dosage Forms and Drug Delivery Systems, 7th ed., Lippencott, Williams & Wilkins (1999); Kibbe et al., Handbook of Pharmaceutical Excipients, 3^{rd} ed., Pharmaceutical Press (2000). Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In pharmaceutical dosage forms, the compositions of the invention can be administered in the form of their pharmaceutically acceptable salts, or they can also be used alone or in appropriate association, as well as in combination, with other pharmaceutically active compounds. The subject compositions are formulated in accordance to the mode of potential administration. Administration of the agents can be achieved in various ways, including oral, buccal, nasal, rectal, parenteral, intraperitoneal, intradermal, transdermal, subcutaneous, intravenous, intra-arterial, intracardiac, intraventricular, intracranial, intratracheal, and intrathecal administration, etc., or otherwise by implantation or inhalation. Thus, the subject compositions can be formulated into preparations in solid, semi-solid, liquid or gaseous forms, such as tablets, capsules, powders, granules, ointments, solutions, suppositories, injections, inhalants and aerosols. The following methods and excipients are merely exemplary and are in no way limiting.

Compositions for oral administration can form solutions, suspensions, tablets, pills, granules, capsules, sustained release formulations, oral rinses, or powders. For oral preparations, the agents, polynucleotides, and polypeptides can be used alone or in combination with appropriate additives, for example, with conventional additives, such as lactose, mannitol, corn starch, or potato starch; with binders, such as crystalline cellulose, cellulose derivatives, acacia, corn starch, or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents.

Suitable excipient vehicles are, for example, water, saline, dextrose, glycerol, ethanol, or the like, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art (Remington, 1985). The composition or formulation to be administered will, in any event, contain a quantity of the agent adequate to achieve the desired state in the subject being treated.

The agents, polynucleotides, and polypeptides can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives. Other formulations for oral or parenteral delivery can also be used, as conventional in the art The antibodies, agents, polynucleotides, and polypeptides can be used in aerosol formulation to be administered via inhalation. The compounds of the present invention can be formulated into pressurized acceptable propellants such as dichlorodifluoromethane, propane, nitrogen, and the like. Further, the agent, polynucleotides, or polypeptide composition may be converted to powder form for administration intranasally or by inhalation, as conventional in the art.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

A polynucleotide, polypeptide, or other modulator, can also be introduced into tissues or host cells by other routes, such as viral infection, microinjection, or vesicle fusion. For example, expression vectors can be used to introduce nucleic acid compositions into a cell as described above. Further, jet injection can be used for intramuscular administration (Furth et al., Anal. Biochem. 205:365 (1992). The DNA can be coated onto gold microparticles, and delivered intradermally by a particle bombardment device, or “gene gun” as described in the literature (Tang et al., Nature 356:152 (1992), where gold microprojectiles are coated with the DNA, then bombarded into skin cells.

Unit dosage forms for oral or rectal administration such as syrups, elixirs, and suspensions can be provided wherein each dosage unit, for example, teaspoonful, tablespoonful, tablet, or suppository, contains a predetermined amount of the composition containing one or more agents. Similarly, unit dosage forms for injection or intravenous administration can comprise the agent(s) in a composition as a solution in sterile water, normal saline or another pharmaceutically acceptable carrier.

2. Active Agents (or Modulators)

The nucleic acid, polypeptide, and modulator compositions of the subject invention find use as therapeutic agents in situations where one wishes to modulate an activity of a polypeptide in a host, particularly the activity of the LRP2, LRP4 or LRP8 polypeptides, or to provide or inhibit the activity at a particular anatomical site. Thus, the compositions are useful in treating disorders associated with an activity of a LRP2, LRP4 or LRP8 polypeptide. The following provides further details of active agents of the present invention.

a) Antisense Oligonucleolides

In certain embodiments of the invention, the active agent is an agent that modulates, and generally decreases or down regulates, the expression of LRP4, LRP8 or LRP2 in a host, i.e., antisense molecules. Anti-sense reagents include antisense oligonucleotides (ODN), i.e., synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g., by reducing the amount of mRNA available for translation, through activation of RNase H, or steric hindrance. One or a combination of antisense molecules can be administered, where a combination can comprise multiple different sequences.

Antisense molecules can be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides can be chemically synthesized by methods known in the art (Wagner et al., Science 260:1510 (1993); Milligan et al., J. Med. Chem. 36:1923 (1993). Antisense oligonucleotides will generally be at least about 7, at least about 12, or at least about 20 nucleotides in length, and not more than about 500, not more than about 50, or not more than about 35 nucleotides in length, where the length is governed by efficiency of inhibition, and specificity, including absence of cross-reactivity, and the like. Short oligonucleotides, of from about 7 to about 8 bases in length, can be strong and selective inhibitors of gene expression (Wagner et al., Nat. Biolechnol. 14:840 (1996).

As an alternative to anti-sense inhibitors, catalytic nucleic acid compounds, e.g., ribozymes, or anti-sense conjugates can be used to inhibit LRP2, LRP4 or LRP8 gene expression. Ribozymes can be synthesized in vitro and administered to the patient, or can be encoded in an expression vector, from which the ribozyme is synthesized in the targeted cell (WO 9523225; Beigelman et al., Nucleic Acids Res. 23:4434 (1995). Examples of oligonucleotides with catalytic activity are described in WO 9506764. Conjugates of anti-sense ODN with a metal complex, e.g., terpyridyl Cu(II), capable of mediating mRNA hydrolysis are described in Bashkin et al., 1995.

b) Interfering RNA

In some embodiments, the active agent is an interfering RNA (RNAi), including dsRNAi. RNA interference provides a method of silencing eukaryotic genes. Use of RNAi to reduce a level of a particular mRNA and/or protein is based on the interfering properties of double-stranded RNA derived from the coding regions of a gene. The technique is an efficient high-throughput method for disrupting gene function (O'Neil et al., Am. J. Pharmacogenomics 1:45 (2001). RNAi can also help identify the biochemical mode of action of a drug and to identify other genes encoding products that can respond or interact with specific compounds.

In one embodiment of the invention, complementary sense and antisense RNAs derived from a substantial portion of the subject polynucleotide are synthesized in vitro. The resulting sense and antisense RNAs are annealed in an injection buffer, and the double-stranded RNA injected or otherwise introduced into the subject, i.e., in food or by immersion in buffer containing the RNA (Gaudilliere et al., J. Biol. Chem. 277:46442 (2002); O'Neil et al., Am. J. Pharmacogenomics 1:45 (2001); WO99/32619). In another embodiment, dsRNA derived from a gene of the present invention is generated in vivo by simultaneously expressing both sense and antisense RNA from appropriately positioned promoters operably linked to coding sequences in both sense and antisense orientations.

c) Peptides and Modified Peptides

In some embodiments of the present invention, the active agent is a peptide. Suitable peptides include peptides of from about 5 amino acids to about 50, from about 6 to about 30, or from about 10 to about 20 amino acids in length. In some embodiments, a peptide has a sequence of from about 7 amino acids to about 45, from about 9 to about 35, or from about 12 to about 25 amino acids of corresponding naturally-occurring protein. In some embodiments, a peptide exhibits one or more of the following activities: inhibits binding of a subject polypeptide to an interacting protein or other molecule; inhibits subject polypeptide binding to a second polypeptide molecule; inhibits a signal transduction activity of a subject polypeptide; inhibits an enzymatic activity of a subject polypeptide; or inhibits a DNA binding activity of a subject polypeptide.

Peptides can include naturally-occurring and non-naturally occurring amino acids. Peptides can comprise D-amino acids, a combination of D- and L-amino acids, and various “designer” amino acids (e.g., β-methyl amino acids, Cα-methyl amino acids, and Nα-methyl amino acids, etc.) to convey special properties. Additionally, peptides can be cyclic. Peptides can include non-classical amino acids in order to introduce particular conformational motifs. Any known non-classical amino acid can be used. Non-classical amino acids include, but are not limited to, 1,2,3,4-tetrahydroisoquinoline-3-carboxylate; (2S,3S)-methylphenylalanine, (2S,3R)-methyl-phenyl alanine, (2R,3 S)-methyl-phenyl alanine and (2R,3R)-methyl-phenyl alanine; 2-aminotetrahydronaphthalene-2-carboxylic acid; hydroxy-1,2,3,4-tetrahydroisoquinoline-3-carboxylate; β-carboline (D and L); HIC (histidine isoquinoline carboxylic acid); and HIC (histidine cyclic urea). Amino acid analogs and peptidomimetics can be incorporated into a peptide to induce or favor specific secondary structures, including, but not limited to, LL-Acp (LL-3-amino-2-propenidone-6-carboxylic acid), a β-turn inducing dipeptide analog; β-sheet inducing analogs; β-turn inducing analogs; α-helix inducing analogs; γ-turn inducing analogs; Gly-Ala turn analogs; amide bond isostere; or tretrazol, and the like.

In addition to the foregoing N-terminal and C-terminal modifications, a peptide or peptidomimetic can be modified with or covalently coupled to one or more of a variety of hydrophilic polymers to increase solubility and circulation half-life of the peptide. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran, and dextran derivatives. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky, Bioconjugate Chem. 6:150 (1995); Monfardini et al., Bioconjugate Chem. 6:62 (1995); U.S. Pat. Nos. 4,640,835; 4,496,689; 4,301,144; 4,670,417; 4,791,192; 4,179,337, or WO 95/34326.

d) Antibodies

The invention provides antibodies that specifically recognize a LRP2, LRP4 or LRP8 polypeptide of the present invention. Antibodies are obtained by immunizing a host animal with peptides, polynucleotides encoding polypeptides, or cells, each comprising all or a portion of the target protein (“immunogen”). Suitable host animals include rodents (e.g., mouse, rat, guinea pig, hamster), cattle (e.g., sheep, pig, cow, horse, goat), cat, dog, chicken, primate, monkey, and rabbit. The origin of the protein immunogen can be any species, including mouse, human, rat, monkey, avian, insect, reptile, or crustacean. The host animal will generally be a different species than the immunogen, e.g., a human protein used to immunize mice. Methods of antibody production are well known in the art (Howard and Bethell, Basic Methods in Antibody Production and Characterization, CRC Press (2000); Harlow et al., Using Antibodies: A Laboratory Manual: Portable Protocol NO. 1, Cold Spring Harbor Laboratory (1998); Harlow and Lane, Antibodies: A Laboratory Manual, Cold Spring Harbor Laboratory (1988).

The immunogen can comprise the complete LRP2, LRP4 or LRP8 protein, or fragments and derivatives thereof, or LRP2, LRP4 or LRP8 proteins expressed on cell surfaces. Immunogens comprise all or a part of one of the LRP2, LRP4 or LRP8 proteins, where these amino acids contain post-translational modifications, such as glycosylation, found on the native target protein. Immunogens comprising protein extracellular domains are produced in a variety of ways known in the art, e.g., expression of cloned genes using conventional recombinant methods, or isolation from tumor cell culture supernatants, etc. The immunogen can also be expressed in vivo from a polynucleotide encoding the immunogenic peptide introduced into the host animal.

Antibody molecules of the invention include immunoglobulin molecules, which are typically composed of heavy and light chains, each of which have constant regions that display similarity with other immunoglobulin molecules and variable regions that convey specificity to particular antigens. Most immunoglobulins can be assigned to classes, e.g., IgG, IgM, IgA, IgE, and IgD, based on antigenic determinants in the heavy chain constant region; each class plays a different role in the immune response.

Antibodies can be used to modulate biological activity, either by increasing or decreasing a stimulation, inhibition, or blockage in the measured activity when compared to a suitable control.

Antibody modulators include antibodies that specifically bind a LRP2, LRP4 or LRP8 polypeptide and activate the polypeptide, such as receptor-ligand binding that initiates signal transduction; antibodies that specifically bind a LRP2, LRP4 or LRP8 polypeptide and inhibit binding of another molecule to the polypeptide, thus preventing activation of a signal transduction pathway; antibodies that bind a LRP2, LRP4 or LRP8 polypeptide to modulate transcription; and antibodies that bind a LRP2, LRP4 or LRP8 polypeptide to modulate translation. An antibody that modulates a biological activity of a LRP2, LRP4 or LRP8 polypeptide or polynucleotide increases or decreases the activity or binding at least about 10%, at least about 15%, at least about 20%, at least about 25%, at least about 50%, at least about 100%, or at least about 2-fold, at least about 5-fold, or at least about 10-fold or more when compared to a suitable control. In one embodiment, a modulator of the invention specifically interferes with the activity of a polypeptide, for example, LRP4, LRP8 or LRP2. More specifically, the antibody specifically binds to the extracellular domain of LRP4, LRP8 or LRP2.

Polyclonal antibodies are prepared by conventional techniques. The method of producing polyclonal antibodies can be varied in some embodiments of the present invention. For example, instead of using a single substantially isolated polypeptide as an immunogen, one may inject a number of different immunogens into one animal for simultaneous production of a variety of antibodies. In addition to protein immunogens, the immunogens can be nucleic acids (e.g., in the form of plasmids or vectors) that encode the proteins, with facilitating agents, such as liposomes, microspheres, etc, or without such agents, such as “naked” DNA.

Antibodies can also be prepared using a library approach. Briefly, mRNA is extracted from the spleens of immunized animals to isolate antibody-encoding sequences. The extracted mRNA may be used to make cDNA libraries. Such a cDNA library may be normalized and subtracted in a manner conventional in the art, for example, to subtract out cDNA hybridizing to mRNA of non-immunized animals. The remaining cDNA may be used to create proteins and for selection of antibody molecules or fragments that specifically bind to the immunogen. The cDNA clones of interest, or fragments thereof, can be introduced into an in vitro expression system to produce the desired antibodies, as described herein.

In a further embodiment, antibodies can be prepared using phage display libraries, conventional in the art. In this method, a collection of bacteriophages displaying antibody properties on their surfaces are made to contact subject polypeptides, or fragments thereof. Bacteriophages displaying antibody properties that specifically recognize the LRP2, LRP4 or LRP8 polypeptides are selected, amplified, for example, in E. coli, and harvested. Such a method typically produces single chain antibodies

Monoclonal antibodies are also produced by conventional techniques, such as fusing an antibody-producing plasma cell with an immortal cell to produce hybridomas. Suitable animals will be used, e.g., to raise antibodies against a mouse polypeptide of the invention, the host animal will generally be a hamster, guinea pig, goat, chicken, or rabbit, and the like. Generally, the spleen and/or lymph nodes of an immunized host animal provide the source of plasma cells, which are immortalized by fusion with myeloma cells to produce hybridoma cells. Culture supernatants from individual hybridomas are screened using standard techniques to identify clones producing antibodies with the desired specificity. The antibody can be purified from the hybridoma cell supernatants or from ascites fluid present in the host by conventional techniques, e.g., affinity chromatography using antigen, e.g., the subject protein, bound to an insoluble support, i.e., protein A sepharose, etc.

The antibody can be produced as a single chain, instead of the normal multimeric structure of the immunoglobulin molecule. Single chain antibodies have been previously described (Jost et al., J. Biol. Chem. 269:26267 (1994). DNA sequences encoding parts of the immunoglobulin, for example, the variable region of the heavy chain and the variable region of the light chain are ligated to a spacer, such as one encoding at least about four small neutral amino acids, i.e., glycine or serine. The protein encoded by this fusion allows the assembly of a functional variable region that retains the specificity and affinity of the original antibody.

The invention also provides intrabodies that are intracellularly expressed single-chain antibody molecules designed to specifically bind and inactivate target molecules inside cells. Intrabodies have been used in cell assays and in whole organisms (Chen et al., Hum. Gene Ther. 5:595 (1994); Hassanzadeh et al., FEBS Lett. 437:75 (1998). Inducible expression vectors can be constructed with intrabodies that react specifically with a protein of the invention. These vectors can be introduced into host cells and model organisms.

The invention also provides “artificial” antibodies, e.g., antibodies and antibody fragments produced and selected in vitro. In some embodiments, these antibodies are displayed on the surface of a bacteriophage or other viral particle, as described above. In other embodiments, artificial antibodies are present as fusion proteins with a viral or bacteriophage structural protein, including, but not limited to, M13 gene III protein. Methods of producing such artificial antibodies are well known in the art (U.S. Pat. Nos. 5,516,637; 5,223,409; 5,658,727; 5,667,988; 5,498,538; 5,403,484; 5,571,698; and 5,625,033). The artificial antibodies, selected for example, on the basis of phage binding to selected antigens, can be fused to a Fc fragment of an immunoglobulin for use as a therapeutic, as described, for example, in U.S. Pat. No. 5,116,964 or WO 99/61630. Antibodies of the invention can be used to modulate biological activity of cells, either directly or indirectly. A subject antibody can modulate the activity of a target cell, with which it has primary interaction, or it can modulate the activity of other cells by exerting secondary effects, i.e., when the primary targets interact or communicate with other cells. The antibodies of the invention can be administered to mammals, and the present invention includes such administration, particularly for therapeutic and/or diagnostic purposes in humans.

Antibodies may be administered by injection systemically, such as by intravenous injection; or by injection or application to the relevant site, such as by direct injection into a tumor, or direct application to the site when the site is exposed in surgery; or by topical application, such as if the disorder is on the skin, for example.

For in vivo use, particularly for injection into humans, in some embodiments it is desirable to decrease the antigenicity of the antibody. An immune response of a recipient against the antibody may potentially decrease the period of time that the therapy is effective. Methods of humanizing antibodies are known in the art. The humanized antibody can be the product of an animal having transgenic human immunoglobulin genes, e.g., constant region genes (e.g., Grosveld and Kolias, Transgenic Animals, 1^{st} ed., Academic Press (1992); Murphy and Carter, Transgenesis Techniques: Principles and Protocols, Humana Press (1993); Pinkert, Transgenic Animal Technology: A Laboratory Handbook, Academic Press, (1994); and International Patent Applications WO 90/10077 and WO 90/04036). Alternatively, the antibody of interest can be engineered by recombinant DNA techniques to substitute the CH1, CH2, CH3, hinge domains, and/or the framework domain with the corresponding human sequence (see, e.g., WO 92/02190). Both polyclonal and monoclonal antibodies made in non-human animals may be “humanized” before administration to human subjects.

Chimeric immunoglobulin genes constructed with immunoglobulin cDNA are known in the art (Liu et al., Proc. Nat. Acad. Sci. USA 84:3439 (1987); Liu et al., J. Immunol. 139:3521 (1987). Messenger RNA is isolated from a hybridoma or other cell producing the antibody and used to produce cDNA. The cDNA of interest can be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes are known in the art (Kabat et al., J. Immunol. 147:1709 (1991). Human C region genes are readily available from known clones. The choice of isotype will be guided by the desired effector functions, such as complement fixation, or antibody-dependent cellular cytotoxicity. IgG1, IgG3 and IgG4 isotypes, and either of the kappa or lambda human light chain constant regions can be used. The chimeric, humanized antibody is then expressed by conventional methods.

Consensus sequences of heavy (“H”) and light (“L”) J regions can be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

A convenient expression vector for producing antibodies is one that encodes a functionally complete human C_{H} or C_{L} immunoglobulin sequence, with appropriate restriction sites engineered so that any V_{H} or V_{L} sequence can be easily inserted and expressed, such as plasmids, retroviruses, YACs, or EBV derived episomes, and the like. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody can be joined to any strong promoter, including retroviral LTRs, e.g., SV-40 early promoter, (Okayama, et al., Mol. Cell. Biol. 3:280 (1983), Rous sarcoma virus LTR (Gorman et al., Proc. Natl. Acad. Sci. USA 79:6777 (1982), and Moloney murine leukemia virus LTR (Grosschedl et al., Cell 41:885 (1985), or native immunoglobulin promoters.

In yet other embodiments, the antibodies can be fully human antibodies. For example, xenogenic antibodies, which are produced in animals that are transgenic for human antibody genes, can be employed. By xenogenic human antibodies is meant antibodies that are fully human antibodies, with the exception that they are produced in a non-human host that has been genetically engineered to express human antibodies. (e.g., WO 98/50433; WO 98,24893 and WO 99/53049).

Antibody fragments, such as Fv, F(ab′)2 and Fab can be prepared by cleavage of the intact protein, e.g., by protease or chemical cleavage. These fragments can include heavy and light chain variable regions. Alternatively, a truncated gene can be designed, e.g., a chimeric gene encoding a portion of the F(ab′)₂ fragment that includes DNA sequences encoding the CH1 domain and hinge region of the H chain, followed by a translational stop codon. The antibodies of the present invention may be administered alone or in combination with other molecules for use as a therapeutic, for example, by linking the antibody to cytotoxic agent, as discussed above, or to a radioactive molecule. Radioactive antibodies that are specific to a cancer cell, disease cell, or virus-infected cell may be able to deliver a sufficient dose of radioactivity to kill such cancer cell, disease cell, or virus-infected cell. The antibodies of the present invention can also be used in assays for detection of the subject polypeptides. In some embodiments, the assay is a binding assay that detects binding of a polypeptide with an antibody specific for the polypeptide; the subject polypeptide or antibody can be immobilized, while the subject polypeptide and/or antibody can be detectably-labeled. For example, the antibody can be directly labeled or detected with a labeled secondary antibody. That is, suitable, detectable labels for antibodies include direct labels, which label the antibody to the protein of interest, and indirect labels, which label an antibody that recognizes the antibody to the protein of interest.

Alternatively, specific-binding pairs may be used, involving, e.g., a second stage antibody or reagent that is detectably-labeled and that can amplify the signal. For example, a primary antibody can be conjugated to biotin, and horseradish peroxidase-conjugated strepavidin added as a second stage reagent. Digoxin and antidigoxin provide another such pair. In other embodiments, the secondary antibody can be conjugated to an enzyme such as peroxidase in combination with a substrate that undergoes a color change in the presence of the peroxidase. The absence or presence of antibody binding can be determined by various methods, including flow cytometry of dissociated cells, microscopy, radiography, or scintillation counting. Such reagents and their methods of use are well known in the art.

e) Peptide Aptamers

Another suitable agent for modulating an activity of a LRP2, LRP4 or LRP8 polypeptide is a peptide aptamer. Peptide aptamers are peptides or small polypeptides that act as dominant inhibitors of protein function. Peptide aptamers specifically bind to target proteins, blocking their functional ability (Kolonin et al., Proc. Natl. Acad. Sci. USA 95:14266 (1998). Due to the highly selective nature of peptide aptamers, they can be used not only to target a specific protein, but also to target specific functions of a given protein (e.g., a signaling function). Further, peptide aptamers can be expressed in a controlled fashion by use of promoters that regulate expression in a temporal, spatial or inducible manner. Peptide aptamers act dominantly, therefore, they can be used to analyze proteins for which loss-of-function mutants are not available.

Peptide aptamers that bind with high affinity and specificity to a target protein can be isolated by a variety of techniques known in the art. Peptide aptamers can be isolated from random peptide libraries by yeast two-hybrid screens (Xu et al., Proc. Natl. Acad. Sci. USA 94:12473 (1997). They can also be isolated from phage libraries (Hoogenboom et al., Immunotechnology 4:1 (1998) or chemically generated peptides/libraries.

### Therapeutic Applications Methods of Use

The instant invention provides various therapeutic methods. In some embodiments, methods of modulating, including increasing and inhibiting, a biological activity of a LRP2, LRP4 or LRP8 protein are provided. In other embodiments, methods of modulating a signal transduction activity of a LRP2, LRP4 or LRP8 protein are provided. In further embodiments, methods of modulating interaction of a LRP2, LRP4 or LRP8 protein with another, interacting protein or other macromolecule (e.g., DNA, carbohydrate, lipid) are provided.

As mentioned above, an effective amount of the active agent (e.g., small molecule, antibody specific for a LRP2, LRP4 or LRP8 polypeptide, a subject polypeptide, or a subject polynucleotide) is administered to the host, where “effective amount” means a dosage sufficient to produce a desired effect or result. In some embodiments, the desired result is at least a reduction in a given biological activity of a subject polypeptide as compared to a control, for example, a decreased level of expression or activity of the subject protein in the individual, or in a localized anatomical site in the individual. In further embodiments, the desired result is at least an increase in a biological activity of a subject polypeptide as compared to a control.

Typically, the compositions of the instant invention will contain from less than about 1% to about 99% of the active ingredient, about 10% to about 90%, or 20% to about 80%, or 30% to about 70%, or 40% to about 60%, or about 50%. Generally, between about 100 mg and about 500 mg will be administered to a child and between about 500 mg and about 5 grams will be administered to an adult.

Other effective dosages can be readily determined by one of ordinary skill in the art through routine trials establishing dose response curves, for example, the amount of agent necessary to increase a level of active subject polypeptide can be calculated from in vitro experimentation. Those of skill will readily appreciate that dose levels can vary as a function of the specific compound, the severity of the symptoms, and the susceptibility of the subject to side effects, and preferred dosages for a given compound are readily determinable by those of skill in the art by a variety of means. For example, in order to calculate the polypeptide, polynucleotide, or modulator dose, those skilled in the art can use readily available information with respect to the amount necessary to have the desired effect, depending upon the particular agent used.

The active agent(s) can be administered to the host via any convenient means capable of resulting in the desired result. Administration is generally by injection and often by injection to a localized area. The frequency of administration will be determined by the care given based on patient responsiveness. For example, the agents may be administered daily, weekly, or as conventionally determined appropriate.

A variety of hosts are treatable according to the subject methods. The host, or patient, may be from any animal species, and will generally be mammalian, e.g., primate sp., e.g., monkeys, chimpanzees, and particularly humans; rodents, including mice, rats and hamsters, guinea pig; rabbits; cattle, including equines, bovines, pig, sheep, goat, canines; felines; etc. Animal models are of interest for experimental investigations, providing a model for treatment of human disease.

The present invention also provides a method for treating diseases including proliferative diseases. The method of the invention provides for treating these diseases with antibodies. This method includes administering antibodies to epitopes of LRP4, LRP8 or LRP2 to a subject. The method of treatment can be for a proliferative disease and in particular, cancer. Cancers that can be treated with antibodies of the invention include splenic, skin, breast, pancreatic, ovarian, kidney, brain, cervical, liver, thyroid, colon, lung, stomach, leukemia and lymphoma.

The antibody is administered locally or systemically. In addition, the antibody is administered intravenously, intra-peritoneally, sub-cutaneously, topically, or transdermally. Furthermore, the antibody is used in a composition with a pharmaceutically acceptable carrier or excipient. A “pharmaceutically acceptable carrier” or “excipient” is intended to include substances that can be co-administered with the compositions of the invention that allows the composition or active molecule therein to perform its intended function. Examples of such carriers include solutions, solvents, buffers, dispersion media, delay agents, emulsions and the like. Further, any other conventional carrier suitable for use with the described antibodies fall within the scope of the instant invention, such as, for example, phosphate buffered saline. The treatment includes administering a therapeutically effective amount of the antibody composition to the subject.

### Proliferative Conditions

In some embodiments, a LRP2, LRP4 or LRP8 protein of the present invention is involved in the control of cell proliferation, and an agent of the invention inhibits undesirable cell proliferation. Such agents are useful for treating disorders that involve abnormal cell proliferation, including, but not limited to, cancer, psoriasis, and scleroderma. Whether a particular agent and/or therapeutic regimen of the invention is effective in reducing unwanted cellular proliferation, e.g., in the context of treating cancer, can be determined using standard methods. Tumors that can be treated using the methods of the instant invention include carcinomas, e.g., colorectal, prostate, breast, bone, kidney, skin, melanoma, ductal, endometrial, stomach or other organ of the gastrointestinal tract, pancreatic, mesothelioma, dysplastic oral mucosa, invasive oral cancer, non-small cell lung carcinoma (“NSCL”), transitional and squamous cell urinary carcinoma; brain cancer and neurological malignancies, e.g., neuroblastoma, glioblastoma, astrocytoma, and gliomas; lymphomas and leukemias such as myeloid leukemia, myelogenous leukemia, hematological malignancies, such as childhood acute leukemia, non-Hodgkin's lymphomas, chronic lymphocytic leukemia, malignant cutaneous T-cell lymphoma, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, and human follicular lymphoma; cancers of the reproductive system, e.g., cervical and ovarian cancers and testicular cancers; liver cancers including hepatocellular carcinoma (“HCC”) and tumors of the biliary duct; multiple myelomas; tumors of the esophageal tract; other lung cancers and tumors including small cell and clear cell; Hodgkin's lymphomas; adenocarcinoma; and sarcomas, including soft tissue sarcomas.

### Pfam

The LRPs of the invention encompass a variety of different types of nucleic acids and polypeptides with different structures and functions. They encode or comprise polypeptides belonging to, inter alia, the Ig protein family (Pfam). The Pfam system is an organization of protein sequence classification and analysis, based on conserved protein domains; it can be publicly accessed in a number of ways, for example, at http://pfam.wustl.edu. Protein domains are portions of proteins that have a tertiary structure and sometimes have enzymatic or binding activities; multiple domains can be connected by flexible polypeptide regions within a protein. Pfam domains can comprise the N-terminus or the C-terminus of a protein, or can be situated at any point in between. The Pfam system identifies protein families based on these domains and provides an annotated, searchable database that classifies proteins into families (Bateman et al., Nucleic Acids Res. 30:276 (2000)).

Molecules of the invention can encode or be comprised of one, or more than one, Pfam. Molecules encompassed by the invention include, the polypeptides and polynucleotides shown in the Sequence Listing and corresponding molecular sequences found at all developmental stages of an organism. Molecules of the invention can comprise genes or gene segments designated by the Sequence Listing, and their gene products, i.e., RNA and polypeptides. They also include variants of those set forth in the Sequence Listing that are present in the normal physiological state, e.g., variant alleles such as SNPs and splice variants, as well as variants that are affected in pathological states, such as disease-related mutations or sequences with alterations that lead to pathology, and variants with conservative amino acid changes.

### Tables

TABLE 1

| | | |
|---|---|---|
| SEQ. ID. NO. 28 | HG1014457P1 | LRP4_EGF1 |

CNVNNGGCAQKCQMVRGAVQCTCHTGYRLTEDGHTC

| | | |
|---|---|---|
| SEQ. ID. NO. 29 | HG1014458P1 | LRP4_EGF2 |

CAMENGGCSHLCLRSPNPSGFSCTCPTGINLLSDGKTC

| | | |
|---|---|---|
| SEQ. ID. NO. 30 | HG1014459P1 | LRP4_EGF3 |

CGSRNGGCSHLCLPRPSGFSCACPTGIQLKGDGKTC

| | | |
|---|---|---|
| SEQ. ID. NO. 31 | HG1014460P1 | LRP4_ldl_recept_a |

LCNGVNDCGDNSDESPQQNCRP

| | | |
|---|---|---|
| SEQ. ID. NO. 32 | HG1014461P1 | LRP4_ldl_recpt_b_1 |

ELVPWSDVTLDRILRANLNGSNVEEVVSTGLESPGGLAVDWV

| | | |
|---|---|---|
| SEQ. ID. NO. 33 | HG1014462P1 | LRP4_ldl_recept_b_2 |

DKLYWTDSGTSRIEVANLDGAHRKVLLWQNLEKPRAIALHPM

| | | |
|---|---|---|
| SEQ. ID. NO. 34 | HG1014463P1 | LRP4_ldl_recept_b_3 |

GTIYWTDWGNTPRIEASSMDGSGRRIIADTHLFWPNGLTIDYA

| | | |
|---|---|---|
| SEQ. ID. NO. 35 | HG1014464P1 | LRP4_ldl_recept_b_4 |

RRMYWVDAKHHVIERANLDGSHRKAVISQGLPHPFAITVFE

| | | |
|---|---|---|
| SEQ. ID. NO. 36 | HG1014465P1 | LRP4_ldl_recept_b_5 |

DHVYWTDVSTDTISRAKWDGTGQEVVVDTSLESPAGLAIDWV

| | | |
|---|---|---|
| SEQ. ID. NO. 37 | HG1014466P1 | LRP4_ldl_recept_b_6 |

NKLYWTDAGTDRIEVANTDGSMRTVLIWENLDRPRDIVVEPM

| | | |
|---|---|---|
| SEQ. ID. NO. 38 | HG1014467P1 | LRP4_ldl_recept_b_7 |

GYMYWTDWGASPKIERAGMDASGRQVIISSNLTWPNGLAIDYG

| | | |
|---|---|---|
| SEQ. ID. NO. 39 | HG1014468P1 | LRP4_ldl_recept_b_8 |

QRLYWADAGMKTIEFAGLDGSKRKVLIGSQLPHPFGLTLY

| | | |
|---|---|---|
| SEQ. ID. NO. 40 | HG1014469P1 | LRP4_ldl_recept_b_9 |

ERIYWTDWQTKSIQSADRLTGLDRETLQENLENLMDIHVFHR

| | | |
|---|---|---|
| SEQ. ID. NO. 41 | HG1014470P1 | LRP4_ldl_recept_b_10 |

GKVYWSDSTLHRISRANLDGSQHEDIITTGLQTTDGLAVDAI

| | | |
|---|---|---|
| SEQ. ID. NO. 42 | HG1014471P1 | LRP4_ldl_recept_b_11 |

RKVYWTDTGTNRIEVGNLDGSMRKVLVWQNLDSPRAIVLYHE

| | | |
|---|---|---|
| SEQ. ID. NO. 43 | HG1014472P1 | LRP4_ldl_recept_b_12 |

GFMYWTDWGENAKLERSGMDGSDRAVLINNNLGWPNGLTVDKA

| | | |
|---|---|---|
| SEQ. ID. NO. 44 | HG1014473P1 | LRP4_ldl_recept_b_13 |

SQLLWADAHTERIEAADLNGANRHTLVSPVQHPYGLTL

| | | |
|---|---|---|
| SEQ. ID. NO. 45 | HG1014474P1 | LRP4_ldl_recept_b_14 |

GKVYYTDVFLDVIRRADLNGSNMETVIGRGLKTTDGLAVDWV

| | | |
|---|---|---|
| SEQ. ID. NO. 46 | HG1014475P1 | LRP4_ldl_recept_b_15 |

RNLYWTDTGRNTIEASRLDGSCRKVLINNSLDEPRAIAVFPR

| | | |
|---|---|---|
| SEQ. ID. NO. 47 | HG1014476P1 | LEP4_ldl_recept_b_16 |

GYLFWTDWGHIAKIERANLDGSERKVLINTDLGWPNGLTLDYD

| | | |
|---|---|---|
| SEQ. ID. NO. 48 | HG1014477P1 | LRP4_ldl_recept_b_17 |

RRIYWVDAHLDRIESADLNGKLRQVLVGHVSHPFALT

| | | |
|---|---|---|
| SEQ. ID. NO. 49 | HG1014478P1 | megalin_EGF_1 |

CSDFNGGCTHECVQEPGGAKCLCPLGFLLANDSKTC

| | | |
|---|---|---|
| SEQ. ID. NO. 50 | HG1014479P1 | megalin_EGF_2 |

CDILGSCSQHCYNMRGSFRCSCDTGYMLESDGRTC

| | | |
|---|---|---|
| SEQ. ID. NO. 51 | HG1014480P1 | megalin_EGF_3 |

CLENNGGCSHLCFALPGLHTPKCDCAFGTLQSDGKNC

| | | |
|---|---|---|
| SEQ. ID. NO. 52 | HG1014481P1 | megalin_EGF_4 |

CTEMPFVCSQKCENVIGSYICKCAPGYLREPDGKTC

| | | |
|---|---|---|
| SEQ. ID. NO. 53 | HG1014482P1 | megalin_EGF_5 |

CMGGNCYFDETDLPKCKCPSGYTGKYC

| | | |
|---|---|---|
| SEQ. ID. NO. 54 | HG1014483P1 | megalin_ldl_recept_a_1 |

QECDSAHFRCGSGHCIPADWRCDGTKDCSDDADEIGCAV

| | | |
|---|---|---|
| SEQ. ID. NO. 55 | HG1014484P1 | megalin_ldl_recept_a_2 |

VTCQQGYFKCQSEGQCIPSSWVCDQDQDCDDGSDERQDCSQ

| | | |
|---|---|---|
| SEQ. ID. NO. 56 | HG1014485P1 | megalin_ldl_recept_a_3 |

STCSSHQITCSNGQCIPSEYRCDHVRDCPDGADENDCQY

| | | |
|---|---|---|
| SEQ. ID. NO. 57 | HG1014486P1 | megalin_ldl_recept_a_4 |

PTCEQLTCDNGACYNTSQKCDWKVDCEDSSDEINCTE

| | | |
|---|---|---|
| SEQ. ID. NO. 58 | HG1014487P1 | megalin_ldl_recept_a_5 |

CLHNEFSCGNGECIPRAYVCDHDNDCQDGSDEHACNY

| | | |
|---|---|---|
| SEQ. ID. NO. 59 | HG1014488P1 | megalin_ldl_recept_a_6 |

PTCGGYQFTCPSGRCIYQNWVCDGEDDCKDNGDEDGCES

| | | |
|---|---|---|
| SEQ. ID. NO. 60 | HG1014489P1 | megalin_ldl_recept_a_7 |

HKCSPREWSCPESGRCISIYKVCDGILDCPGREDENNTSTGKYCSN

| | | |
|---|---|---|
| SEQ. ID. NO. 61 | HG1014490P1 | megalin_ldl_recept_a_8 |

EQCGLFSFPCKNGRCVPNYYLCDGVDDCHDNSDEQLCGT

| | | |
|---|---|---|
| SEQ. ID. NO. 62 | HG1014491P1 | megalin_ldl_recept_a_9 |

NTCSSSAFTCGHGECIPAHWRCDKRNDCVDGSDEHNCPT

| | | |
|---|---|---|
| SEQ. ID. NO. 63 | HG1014492P1 | megalin_ldl_recept_a_10 |

ASCLDTQYTCDNHQCISKNWVCDTDNDCGDGSDEKNCNS

| | | |
|---|---|---|
| SEQ. ID. NO. 64 | HG1014493P1 | megalin_ldl_recept_a_11 |

ETCQPSQFNCPNHRCIDLSFVCDGDKDCVDGSDEVGCV

| | | |
|---|---|---|
| SEQ. ID. NO. 65 | HG1014494P1 | megalin_ldl_recept_a_12 |

LNCTASQFKCASGDKCIGVTNRCDGVFDCSDNSDEAGCPT

| | | |
|---|---|---|
| SEQ. ID. NO. 66 | HG1014495P1 | megalin_ldl_recept_a_13 |

GMCHSDEFQCQEDGICIPNFWECDGHPDCLYGSDEHNACVP

| | | |
|---|---|---|
| SEQ. ID. NO. 67 | HG1014496P1 | megalin_ldl_recept_a_14 |

KTCPSSYFHCDNGNCIHRAWLCDRDNDCGDMSDEKDCPT

| | | |
|---|---|---|
| SEQ. ID. NO. 68 | HG1014497P1 | megalin_ldl_recept_a_15 |

FRCPSWQWQCLGHNICVNLSVVCDGIFDCPNGTDESPLGNG

| | | |
|---|---|---|
| SEQ. ID. NO. 69 | HG1014498P1 | megalin_ldl_recept_a_16 |

ERCGASSFTCSNGRCISEEWKCDNDNDCGDGSDEMESVCAL

| | | |
|---|---|---|
| SEQ. ID. NO. 70 | HG1014499P1 | megalin_ldl recept_a_17 |

HTCSPTAFTCANGRCVQYSYRCDYYNDCGDGSDEAGCLF

| | | |
|---|---|---|
| SEQ. ID. NO. 71 | HG1014500P1 | megalin_ldl_recept_a_18 |

RDCNATTEFNCNNRRCIPREFICNGVDNCHDNNTSDEKNCPD

| | | |
|---|---|---|
| SEQ. ID. NO. 72 | HG1014501P1 | megalin_ldl_recept_a_19 |

RTCQSGYTKCHNSNICIPRVYLCDGDNDCGDNSDENPTYCTT

| | | |
|---|---|---|
| SEQ. ID. NO. 73 | HG1014502P1 | megalin_ldl_recept_a_20 |

HTCSSSEFQCASGRCIPQHWYCDQETDCFDASDEPASCGH

| | | |
|---|---|---|
| SEQ. ID. NO. 74 | HG1014503P1 | megalin_ldl_recept_a_21 |

RTCLADEFKCDGGRCIPSEWICDGDNDCGDMSDEDKRHQCQN

| | | |
|---|---|---|
| SEQ. ID. NO. 75 | HG1014504P1 | megalin_ldl_recept_a_22 |

QNCSDSEFLCVNDRPPDRRCIPQSWVCDGDVDCTDGYDENQNCTR

| | | |
|---|---|---|
| SEQ. ID. NO. 76 | HG1014505P1 | megalin_ldl_recept_a_23 |

RTCSENEFTCGYGLCIPKIFRCDRHNDCGDYSDERGCLY

| | | |
|---|---|---|
| SEQ. ID. NO. 77 | HG1014506P1 | megalin_ldl_recept_a_24 |

QTCQQNQFTCQNGRCISKTFVCDEDNDCGDGSDELMHLCHT

| | | |
|---|---|---|
| SEQ. ID. NO. 78 | HG1014507P1 | megalin_ldl_recept_a_25 |

PTCPPHEFKCDNGRCIEMMKLCNHLDDCLDNSDEKGCGI

| | | |
|---|---|---|
| SEQ. ID. NO. 79 | HG1014508P1 | megalin_ldl_recept_a_26 |

PMCSSTQFLCANNEKCIPIWWKCDGQKDCSDGSDELALCPQ

| | | |
|---|---|---|
| SEQ. ID. NO. 80 | HG1014509P1 | megalin_ldl_recept_a_27 |

RFCRLGQFQCSDGNCTSPQTLCNAHQNCPDGSDEDRLLCEN

| | | |
|---|---|---|
| SEQ. ID. NO. 81 | HG1014510P1 | megalin_ldl_recept_a_28 |

HHCDSNEWQCANKRCIPESWQCDTFNDCEDNSDEDSSHCAS

| | | |
|---|---|---|
| SEQ. ID. NO. 82 | HG1014511P1 | megalin_ldl_recept_a_29 |

RTCRPGQFRCANGRCIPQAWKCDVDNDCGDHSDEPIEECMS

| | | |
|---|---|---|
| SEQ. ID. NO. 83 | HG1014512P1 | megalin_ldl_recept_a_30 |

EFSCKTNYRCIPKWAVCNGVDDCRDNSDEQGCEE

| | | |
|---|---|---|
| SEQ. ID. NO. 84 | HG1014513P1 | megalin_ldl_recept_a_31 |

RTCHPVGDFRCKNHHCIPLRWQCDGQNDCGDNSDEENCAP

| | | |
|---|---|---|
| SEQ. ID. NO. 85 | HG1014514P1 | megalin_ldl_recept_a_32 |

RECTESEFRCVNQQCIPSRWICDHYNDCGDNSDERDCEM

| | | |
|---|---|---|
| SEQ. ID. NO. 86 | HG1014515P1 | megalin_ldl_recept_a_33 |

RTCHPEYFQCTSGHCVHSELKCDGSADCLDASDEADCPT

| | | |
|---|---|---|
| SEQ. ID. NO. 87 | HG1014516P1 | megalin_ldl_recept_a_34 |

AYCQATMFECKNHVCIPPYWICDGDDDCGDGSDEELHLCLD

| | | |
|---|---|---|
| SEQ. ID. NO. 88 | HG1014517P1 | megalin_ldl_recept_a_35 |

VPCNSPNRFRCDNNRCIYSHEVCNGVDDCGDGTDETEEHCRK

| | | |
|---|---|---|
| SEQ. ID. NO. 89 | HG1014518P1 | megalin_ldl_recept_a_36 |

KPCTEYEYKCGNGHCIPHDNVCDDADDCGDWSDELGCNK

| | | |
|---|---|---|
| SEQ. ID. NO. 90 | HG1014519P1 | megalin_ldl_recept_b_1 |

QRVFWTDTVQNKVFSVDINGLNIQEVLNVSVETPENLAVDWV

| | | |
|---|---|---|
| SEQ. ID. NO. 91 | HG1014520P1 | megalin_ldl_recept_b_2 |

NKIYLVETKVNRIDMVNLDGSYRVTLITENLGHPRGIAVDPT

| | | |
|---|---|---|
| SEQ. ID. NO. 92 | HG1014521P1 | megalin_ldl_recept_b_3 |

GYLFFSDWESLSGEPKLERAFMDGSNRKDLVKTKLGWPAGVTLDMI

| | | |
|---|---|---|
| SEQ. ID. NO. 93 | HG1014522P1 | megalin_ldl_recept_b_4 |

STIFFSDMSKHMIFKQRIDGTGREILAANRVENVESLAFDWI

| | | |
|---|---|---|
| SEQ. ID. NO. 94 | HG1014523P1 | megalin_ldl_recept_b_5 |

KNLYWTDSHYKSISVMRLADKTRRTVVQYLNNPRSVVVHPF

| | | |
|---|---|---|
| SEQ. ID. NO. 95 | HG1014524P1 | megalin_ldl_recept_b_6 |

GYLFFTDWFRPAKIMRAWSDGSHLLPVINTTLGWPNGLAIDWA

| | | |
|---|---|---|
| SEQ. ID. NO. 96 | HG1014525P1 | megalin_ldl_recept_b_7 |

GRIFWSDATQGKTWSAFQNGTDRRVVFDSSIILTETIAIDWV

| | | |
|---|---|---|
| SEQ. ID. NO. 97 | HG1014526P1 | megalin_ldl_recept_b_8 |

RNLYWTDYALETIEVSKIDGSHRTVLISKNLTNPRGLALDPR

| | | |
|---|---|---|
| SEQ. ID. NO. 98 | HG1014527P1 | megalin_ldl_recept_b_9 |

HLLFWSDWGHHPRIERASMDGSMRTVIVQDKIFWPCGLTIDYP

| | | |
|---|---|---|
| SEQ. ID. NO. 99 | HG1014528P1 | megalin_ldl_recept_b_10 |

GKLYWSDQGTDSGVPAKIASANMDGTSVKTLFTGNLEHLJECVTLDIE

| | | |
|---|---|---|
| SEQ. ID. NO. 100 | HG1014529P1 | megalin_ldl_recept_b_11 |

QKLYWAVTGRGVIERGNVDGTDRMILVHQLSHPWGIAIVH

| | | |
|---|---|---|
| SEQ. ID. NO. 101 | HG1014530P1 | megalin_ldl_recept_b_12 |

RYLFWADYGQRPKIERSFLDCTNRTVLVSEGIVTPRGLAVDRS

| | | |
|---|---|---|
| SEQ. ID. NO. 102 | HG1014531P1 | megalin_ldl_recept_a_49 |

GYLYWADWDTHAKIERATLGGNFRVPIVNSSLVMPSGLTLDYE

| | | |
|---|---|---|
| SEQ. ID. NO. 103 | HG1014532P1 | megalin_ldl_recept_a_50 |

DLLYWVDASLQRIERSTLTGVDREVIVNAAVIIAFGLTLY

| | | |
|---|---|---|
| SEQ. ID. NO. 104 | HG1014533P1 | megalin_ldl_recept_b_15 |

ERLYWIDTQRQVIERWFLNKTNKETIINHIRLPAAESLAVDWV

| | | |
|---|---|---|
| SEQ. ID. NO. 105 | HG1014534P1 | megalin_ldl_recept_b_16 |

RKLYWLDARLDGLFVSDLNGGHRRMLAQHCVDANNTFCFDNPRGLALHPQ

| | | |
|---|---|---|
| SEQ. ID. NO. 106 | HG1014535P1 | megalin_ldl_recept_b_17 |

GYLYWADWGHRAYIGRVGMDGTNKSVIISTKLEWPNGITIDYT

| | | |
|---|---|---|
| SEQ. ID. NO. 107 | HG1014536P1 | megalin_ldl_recept_b_18 |

DLLYWADAHLGYIEYSDLEGHHRHTVYDGALPHPFAITIFE

| | | |
|---|---|---|
| SEQ. ID. NO. 108 | HG1014537P1 | megalin_ldl_recept_b_19 |

DTIYWTDWNTRTVEXGNKYDGSNRQTLVNTTHRPFDIHVYHP

| | | |
|---|---|---|
| SEQ. ID. NO. 109 | HG1014538P1 | megalin_ldl_recept_b_20 |

RHIYWSDVKNKRIEVAKLDGRYRKWLISTDLDQPAAIAVNPK

| | | |
|---|---|---|
| SEQ. ID. NO. 110 | HG1014539P1 | megalin_ldl_recept_b_21 |

GLMFWTDWGKEPKIESAWMNGEDRNILVFEDLGWPTGLSIDYL

| | | |
|---|---|---|
| SEQ. ID. NO. 111 | HG1014540P1 | megalin_ldl_recept_b_22 |

DRIYWSDFKEDVIETIKYDGTDRRVIAKEA

| | | |
|---|---|---|
| SEQ. ID. NO. 112 | HG1014541P1 | LRP8_EGF |

CLHNNGGCSHICTDLKIGFECTCPAGFQLLDQKTC

| | | |
|---|---|---|
| SEQ. ID. NO. 113 | HG1014542P1 | LRP8_ldl_recpt_a_1_v2 |

KDCEKDQFQCRNERCIPSVWRCDEDDDCLDHSDEDDCPK

| | | |
|---|---|---|
| SEQ. ID. NO. 114 | HG1014543P1 | LRP8_ldl_recpt_a_2 |

GTCRGDEFQCGDGTCVLAIKHCNQEQDCPDGSDEAGCLQ

| | | |
|---|---|---|
| SEQ. ID. NO. 115 | HG1014544P1 | LRP8_ldl_recpt_a_1 |

KECEKDQFQCRNERCIPSVWRCDEDDDCLDHSDEDDCPK

| | | |
|---|---|---|
| SEQ. ID. NO. 116 | HG1014545P1 | LRP8_ldl_recpt_a_3 |

KTCADSDFTCDNGHCIHERWKCDGEEECPDGSDESEATCTK

| | | |
|---|---|---|
| SEQ. ID. NO. 117 | HG1014546P1 | LRP8_ldl_recpt_a_4 |

SHKCVPASWRCDGEKDCEGGADEAGCAT

| | | |
|---|---|---|
| SEQ. ID. NO. 118 | HG1014547P1 | LRP8_ldl_recpt_a_5 |

CAPHEFQCGNRSCLAAVFVCDGDDDCGDGSDERGCAD

| | | |
|---|---|---|
| SEQ. ID. NO. 119 | HG1014549P1 | LRP8_ldl_recpt_a_7 |

AACATVSQFACRSGECVHLGWRCDGDRDCKDKSDEADCPL

| | | |
|---|---|---|
| SEQ. ID. NO. 120 | HG1014549P1 | LRP8_ldl_recpt_b_1 |

NRIYWCDLSYRKIYSAYMDKASDPKEQEVLIDEQLHSPEGLAVDWV

| | | |
|---|---|---|
| SEQ. ID. NO. 121 | HG1014550P1 | LRP8_ldl_recpt_b_1 |

NRIYWCDLSYRKIYSAYMDKASDPKEREVLIDEQLHSPEGLAVDWV

| | | |
|---|---|---|
| SEQ. ID. NO. 122 | HG1014551P1 | LRP8_ldl_recpt_b_1_v2 |

NRIYWCDLSYRKIYSAYMDKASDPKEQEVLIDEQLHSPEGLAVDWV

| | | |
|---|---|---|
| SEQ. ID. NO. 123 | HG1014552P1 | LRP8_ldl_recpt_b_2 |

KHIYWTDSGNKTISVATVDGGRRRTLFSRNLSEPRAIAVDPL

| | | |
|---|---|---|
| SEQ. ID. NO. 124 | HG1014553P1 | LRP8_ldl_recpt_b_3 |

GFMYWSDWGDQAKIEKSGLNGVDRQTLVSDNIEWPNGITLDLL

| | | |
|---|---|---|
| SEQ. ID. NO. 125 | HG1014554P1 | LRP8_ldl_recpt_b_4 |

QRLYWVDSKLHQLSSIDFSGGNRKTLISSTDFLSHPFGIAVFE

| | | |
|---|---|---|
| SEQ. ID. NO. 126 | HG2014554P1 | LRP8_ldl_recpt_b_5 |

DKVFWTDLENEAIFSANRLNGLEISILAENLNNPHDIVIFHE

TABLE 2

| Internal | SEQ.ID. | SEQ.ID. | SEQ.ID. | Classifi- | | |
|---|---|---|---|---|---|---|
| ID No. | NO.(N1) | NO.(P1) | NO.(NO) | cation | Source (aa) | Source (nt) |
| HG1014438 | SEQ.ID. | SEQ.ID. | SEQ.ID. | LRP8 | NP_059992 | NM_017522 |
| | NO. 1 | NO. 10 | NO. 19 | | | |
| HG1014439 | SEQ.ID. | SEQ.ID. | SEQ.ID. | LRP8 | NP_150643 | NM_033300 |
| | NO. 2 | NO. 11 | NO. 20 | | | |
| HG1014440 | SEQ.ID. | SEQ.ID. | SEQ.ID. | LRP8 | NP_004622 | NM_004631 |
| | NO. 3 | NO. 12 | NO. 21 | | | |
| HG1014441 | SEQ.ID. | SEQ.ID. | SEQ.ID. | LRP8 | NP_004622_mod_by_CLN00147603 | NP_004622_mod_by_CLN001 |
| | NO. 4 | NO. 13 | NO. 22 | | | 47603 |
| HG1014442 | SEQ.ID. | SEQ.ID. | SEQ.ID. | LRP8 | H020C00-A-000 | H020C00-A- |
| | NO. 5 | NO. 14 | NO. 23 | | 1_A03.abl_5pclone | 0001_A03.abl_5pclone |
| HG1014443 | SEQ.ID. | SEQ.ID. | SEQ.ID. | LRP8 | PLT00006704_G09.abl3pclone | PLT00006704_G09.abl_3pclone |
| | NO. 6 | NO. 15 | NO. 24 | | | |
| HG1014444 | SEQ.ID. | SEQ.ID. | SEQ.ID. | megalin | NP_004516 | NM_004525 |
| | NO. 7 | NO. 16 | NO. 25 | | | |
| HG1014445 | SEQ.ID. | SEQ.ID. | SEQ.ID. | LRP4 | 22065231 | 22065230 |
| | NO. 8 | NO. 17 | NO. 26 | | | |
| HG1014446 | SEQ.ID. | SEQ.ID. | SEQ.ID. | LRP4 | 3449306 | 3449305 |
| | NO. 9 | NO. 18 | NO. 27 | | | |
| HG1014457 | | SEQ.ID. | | LRP4 | LRP4_EGF1 | |
| | | NO. 28 | | | | |
| HG1014458 | | SEQ.ID. | | LRP4 | LRP4_EGF2 | |
| | | NO. 29 | | | | |
| HG1014459 | | SEQ.ID. | | LRP4 | LRP4_EGF3 | |
| | | NO. 30 | | | | |
| HG1014460 | | SEQ.ID. | | LRP4 | LRP4_ldlrecept_a | |
| | | NO. 31 | | | | |
| HG1014461 | | SEQ.ID. | | LRP4 | LRP4_ldl_recept_b_1 | |
| | | NO. 32 | | | | |
| HG1014462 | | SEQ.ID. | | LRP4 | LRP4_ldl_receptb_2 | |
| | | NO. 33 | | | | |
| HG1014463 | | SEQ.ID. | | LRP4 | LRP4ldl_recept_b_3 | |
| | | NO. 34 | | | | |
| HG1014464 | | SEQ.ID. | | LRP4 | LRP4_ldl_recept_b_4 | |
| | | NO. 35 | | | | |
| HG1014465 | | SEQ.ID. | | LRP4 | LRP4_ldl_recept_b_5 | |
| | | NO. 36 | | | | |
| HG1014466 | | SEQ.ID. | | LRP4 | LRP4_ldl_recept_b_6 | |
| | | NO. 37 | | | | |
| HG1014467 | | SEQ.ID. | | LRP4 | LRP4_ldl_recept_b_7 | |
| | | NO. 38 | | | | |
| HG1014468 | | SEQ.ID. | | LRP4 | LRP4_ldl_recept_b_8 | |
| | | NO. 39 | | | | |
| HG1014469 | | SEQ.ID. | | LRP4 | LRP4_ldl_recept_b_9 | |
| | | NO. 40 | | | | |
| HG1014470 | | SEQ.ID. | | LRP4 | LRP4_ldl_recept_b_10 | |
| | | NO. 41 | | | | |
| HG1014471 | | SEQ.ID. | | LRP4 | LRP4_ldl_recept_b_11 | |
| | | NO. 42 | | | | |
| HG1014472 | | SEQ.ID. | | LRP4 | LRP4_ldl_recept_b_12 | |
| | | NO. 43 | | | | |
| HG1014473 | | SEQ.ID. | | LRP4 | LRP4_ldl_recept_b_13 | |
| | | NO. 44 | | | | |
| HG1014474 | | SEQ.ID. | | LRP4 | LRP4_ldl_recept_b_14 | |
| | | NO. 45 | | | | |
| HG1014475 | | SEQ.ID. | | LRP4 | LRP4_ldl_recept_b_15 | |
| | | NO. 46 | | | | |
| HG1014476 | | SEQ.ID. | | LRP4 | LRP4_ldl_recept_b_16 | |
| | | NO. 47 | | | | |
| HG1014477 | | SEQ.ID. | | LRP4 | LRP4_ldl_recept_b_17 | |
| | | NO. 48 | | | | |
| HG1014478 | | SEQ.ID. | | megalin | megalin_EGF_1 | |
| | | NO. 49 | | | | |
| HG1014479 | | SEQ.ID. | | megalin | megalin_EGF_2 | |
| | | NO. 50 | | | | |
| HG1014480 | | SEQ.ID. | | megalin | megalin_EGF_3 | |
| | | NO. 51 | | | | |
| HG1014481 | | SEQ.ID. | | megalin | megalin_EGF_4 | |
| | | NO. 52 | | | | |
| HG1014482 | | SEQ.ID. | | megalin | megalin_EGF_5 | |
| | | NO. 53 | | | | |
| HG1014483 | | SEQ.ID. | | megalin | megalin_ldl_recept_a_1 | |
| | | NO. 54 | | | | |
| HG1014484 | | SEQ.ID. | | megalin | megalin_ldl_recept_a_2 | |
| | | NO. 55 | | | | |
| HG1014485 | | SEQ.ID. | | megalin | megalin_ldl_recept_a_3 | |
| | | NO. 56 | | | | |
| HG1014486 | | SEQ.ID. | | megalin | megalin_ldl_recept_a_4 | |
| | | NO. 57 | | | | |
| HG1014487 | | SEQ.ID. | | megalin | megalin_ldl_recept_a_5 | |
| | | NO. 58 | | | | |
| HG1014488 | | SEQ.ID. | | megalin | megalin_ldl_recept_a_6 | |
| | | NO. 59 | | | | |
| HG1014489 | | SEQ.ID. | | megalin | megalin_ldl_recept_a_7 | |
| | | NO. 60 | | | | |
| HG1014490 | | SEQ.ID. | | megalin | megalin_ldl_recept_a_8 | |
| | | NO. 61 | | | | |
| HG1014491 | | SEQ.ID. | | megalin | megalin_ldl_recept_a.9 | |
| | | NO. 62 | | | | |
| HG1014492 | | SEQ.ID. | | megalin | megalin_ldl_recept_a_10 | |
| | | NO. 63 | | | | |
| HG1014493 | | SEQ.ID. | | megalin | megalin_ldl_recept_a_11 | |
| | | NO. 64 | | | | |
| HG1014494 | | SEQ.ID. | | megalin | megalin_ldl_recept_a_12 | |
| | | NO. 65 | | | | |
| HG1014495 | | SEQ.ID. | | megalin | megalin_ldl_recept_a_13 | |
| | | NO. 66 | | | | |
| HG1014496 | | SEQ.ID. | | megalin | megalin_ldl_recept_a_14 | |
| | | NO. 67 | | | | |
| HG1014497 | | SEQ.ID. | | megalin | megalin_ldl_recept_a_15 | |
| | | NO. 68 | | | | |
| HG1014498 | | SEQ.ID. | | megalin | megalin_ldl_recept_a_16 | |
| | | NO. 69 | | | | |
| HG1014499 | | SEQ.ID. | | megalin | megalin_ldl_recept_a_17 | |
| | | NO. 70 | | | | |
| HG1014500 | | SEQ.ID. | | megalin | megalin_ldl_recept_a_18 | |
| | | NO. 71 | | | | |
| HG1014501 | | SEQ.ID. | | megalin | megalin_ldl_recept_a_19 | |
| | | NO. 72 | | | | |
| HG1014502 | | SEQ.ID. | | megalin | megalin_ldl_recept_a_20 | |
| | | NO. 73 | | | | |
| HG1014503 | | SEQ.ID. | | megalin | megalin_ldl_recepta_21 | |
| | | NO. 74 | | | | |
| HG1014504 | | SEQ.ID. | | megalin | megalin_ldl_recept_a_22 | |
| | | NO. 75 | | | | |
| HG1014505 | | SEQ.ID. | | megalin | megalin_ldl_recept_a_23 | |
| | | NO. 76 | | | | |
| HG1014506 | | SEQ.ID. | | megalin | megalin_ldl_recept_a_24 | |
| | | NO. 77 | | | | |
| HG1014507 | | SEQ.ID. | | megalin | megalin_ldl_recept_a_25 | |
| | | NO. 78 | | | | |
| HG1014508 | | SEQ.ID. | | megalin | megalin_ldl_recept_a_26 | |
| | | NO. 79 | | | | |
| HG1014509 | | SEQ.ID. | | megalin | megalin_ldl_recept_a27 | |
| | | NO. 80 | | | | |
| HG1014510 | | SEQ.ID. | | megalin | megalin_ldl_recept_a_28 | |
| | | NO. 81 | | | | |
| HG1014511 | | SEQ.ID. | | megalin | megalin_ldl_recept_a_29 | |
| | | NO. 82 | | | | |
| HG1014512 | | SEQ.ID. | | megalin | megalin_ldl_recept_a_30 | |
| | | NO. 83 | | | | |
| HG1014513 | | SEQ.ID. | | megalin | megalin_ldl_recept_a_31 | |
| | | NO. 84 | | | | |
| HG1014514 | | SEQ.ID. | | megalin | megalin_ldl_recept_a_32 | |
| | | NO. 85 | | | | |
| HG1014515 | | SEQ ID. | | megalin | megalin_ldl_recept_a_33 | |
| | | NO. 86 | | | | |
| HG1014516 | | SEQ.ID. | | megalin | megalin_ldl_recept_a_34 | |
| | | NO. 87 | | | | |
| HG1014517 | | SEQ.ID. | | megalin | megalin_ldl_recept_a_35 | |
| | | NO. 88 | | | | |
| HG1014518 | | SEQ.ID. | | megalin | megalin_ldl_recepta_36 | |
| | | NO. 89 | | | | |
| HG1014519 | | SEQ.ID. | | megalin | megalin_ldl_recept_b_1 | |
| | | NO. 90 | | | | |
| HG1014520 | | SEQ.ID. | | megalin | megalin_ldl_recept_b_2 | |
| | | NO. 91 | | | | |
| HG1014521 | | SEQ.ID. | | megalin | megalin_ldl_recept_b_3 | |
| | | NO. 92 | | | | |
| HG1014522 | | SEQ.ID. | | megalin | megalin_ldl_recept_b_4 | |
| | | NO. 93 | | | | |
| HG1014523 | | SEQ.ID. | | megalin | megalin_ldl_recept_b.5 | |
| | | NO. 94 | | | | |
| HG1014524 | | SEQ.ID. | | megalin | megalin_ldl_recept_b_6 | |
| | | NO. 95 | | | | |
| HG1014525 | | SEQ.ID. | | megalin | megalin_ldl_recept_b_7 | |
| | | NO. 96 | | | | |
| HG1014526 | | SEQ.ID. | | megalin | megalin_ldl_recept_b_8 | |
| | | NO. 97 | | | | |
| HG1014527 | | SEQ.ID. | | megalin | megalin_ldl_recept_b_9 | |
| | | NO. 98 | | | | |
| HG1014528 | | SEQ.ID. | | megalin | megalin_ldl_recept_b_10 | |
| | | NO. 99 | | | | |
| HG1014529 | | SEQ.ID. | | megalin | megalin_ldl_recept_b_11 | |
| | | NO. 100 | | | | |
| HG1014530 | | SEQ.ID. | | megalin | megalin_ldl_recept_b_12 | |
| | | NO. 101 | | | | |
| HG1014531 | | SEQ.ID. | | megalin | megalin_ldl_recept_b_13 | |
| | | NO. 102 | | | | |
| HG1014532 | | SEQ.ID. | | megalin | megalin_ldl_recept_b_14 | |
| | | NO. 103 | | | | |
| HG1014533 | | SEQ.ID. | | megalin | megalin_ldl_recept_b_15 | |
| | | NO. 104 | | | | |
| HG1014534 | | SEQ.ID. | | megalin | megalin_ldl_recept_b_16 | |
| | | NO. 105 | | | | |
| HG1014535 | | SEQ.ID. | | megalin | megalin_ldl_recept_b_17 | |
| | | NO. 106 | | | | |
| HG1014536 | | SEQ.ID. | | megalin | megalin_ldl_recept_b_18 | |
| | | NO. 107 | | | | |
| HG1014537 | | SEQ.ID. | | megalin | megalin_ldl_recept_b_19 | |
| | | NO. 108 | | | | |
| HG1014538 | | SEQ.ID. | | megalin | megalin_ldl_recept_b_20 | |
| | | NO. 109 | | | | |
| HG1014539 | | SEQ.ID. | | megalin | megalin_ldl_recept_b_21 | |
| | | NO. 110 | | | | |
| HG1014540 | | SEQ.ID. | | megalin | megalin_ldl_recept_b_22 | |
| | | NO. 111 | | | | |
| HG1014541 | | SEQ.ID. | | LRP8 | LRP8_EGF | |
| | | NO. 112 | | | | |
| HG1014542 | | SEQ.ID. | | LRP8 | LRP8_ldl_recept_a_1_v2 | |
| | | NO. 113 | | | | |
| HG1014543 | | SEQ.ID. | | LRP8 | LRP8_ldl_recept_a_2 | |
| | | NO. 114 | | | | |
| HG1014544 | | SEQ.ID. | | LRP8 | LRP8_ldl_recept_a_1 | |
| | | NO. 115 | | | | |
| HG1014545 | | SEQ.ID. | | LRP8 | LRP8_ldl_recept_a_3 | |
| | | NO. 116 | | | | |
| HG1014546 | | SEQ.ID. | | LRP8 | LRP8_ldl_recept_a_4 | |
| | | NO. 117 | | | | |
| HG1014547 | | SEQ.ID. | | LRP8 | LRP8_ldl_recept_a_5 | |
| | | NO. 118 | | | | |
| HG1014548 | | SEQ.ID. | | LRP8 | LRP8_ldl_recept_a_6 | |
| | | NO. 119 | | | | |
| HG1014549 | | SEQ.ID. | | LRP8 | LRP8_ldl_recept_a_7 | |
| | | NO. 120 | | | | |
| HG1014550 | | SEQ.ID. | | LRP8 | LRP8_ldl_recept_b_1 | |
| | | NO. 121 | | | | |
| HG1014551 | | SEQ.ID. | | LRP8 | LRP8_ldl_recept_b_1_v2 | |
| | | NO. 122 | | | | |
| HG1014552 | | SEQ.ID. | | LRP8 | LRP8_ldl_recept_b_2 | |
| | | NO. 123 | | | | |
| HG1014553 | | SEQ.ID. | | LRP8 | LRP8_ldl_recept_b_3 | |
| | | NO. 124 | | | | |
| HG1014554 | | SEQ.ID. | | LRP8 | LRPB_ldl_recept_b_4 | |
| | | NO. 125 | | | | |
| HG1014555 | | SEQ.ID. | | LRP8 | LRP8_ldl_recept_b_5 | |
| | | NO. 126 | | | | |

TABLE 3
CLUSTAL W (1.8) multiple sequence alignment

| | |
|---|---|
| megalin_{—} | CDILG-SCSQHCY--NMRGSFRCSCDTGYMLESDGRTC |
| EGF_2 | |
| megalin_{—} | CTEMPFVCSQKCE--NVIGSYICKCAPGYLREPDGKTC |
| EGF_4 | |
| LRP_4EGF1 | CNVNNGGCAQKCQ--MVRGAVQCTCHTGYRLTEDGHTC |
| LRP4_EGF2 | CAMENGGCSHLCLRSPNPSGFSCTCPTGINLLSDGKTC |
| LRP4_EGF3 | CGSRNGGCSHLCL--PRPSGFSCACPTGIQLKGDGKTC |
| megalin_{—} | CSDFNGGCTHECV--QEPFGAKCLCPLGFLLANDSKTC |
| EGF1 | |
| megalin_{—} | CLENNGGCSHLCFALPGLHTPKCDCAFG-TLQSDGKNC |
| EGF_3 | |
| LRP8_EGF | CLHNNGGCSHICT--DLKIGFECTCPAGFQLL-DQKTC |
| megalin_{—} | CMH-GGNCYFDET--DLP---KCKCPSGYT----GKYC |
| EGF_5 | |

TABLE 4
CLUSTAL W (1.8) multiple sequence alignment

| | |
|---|---|
| megalin_ldlra_5 | ---CL-HNEFSCG-N---GE-----CIPRAYVCDHDNDCQDG--SDE---HA--CNY--- |
| megalin_ldlra_36 | -KPCT-EYEYKCG-N---GH-----CIPHDNVCDDADDCGDW--SDE---LG--CNK--- |
| LRP8ldlra6 | -PACG-PREFRCG-GDGGGA-----CIPERWVCDRQFDCEDR--SDE---AAELCGR--- |
| LRP4_ldlra | -------------------------------LCNGVNDCGDN--SDESPQQN--CRP--- |
| megalin_ldlra_19 | -RTCQ-SGYTKCH-NS--NI-----CIPRVYLCDGDNDCGDN--SDENP-TY--CTT--- |
| megalin_ldlra_31 | -RTCHPVGDFRCK-N---HH-----CIPLRWQCDGQNDCGDN--SDE---EN--CAP--- |
| megalin_ldra_32 | -RECT-ESEFRCV-N---QQ-----CIPSRWICDHYNDCGDN--SDE---RD--CEM--- |
| megalin_ldra_29 | -RTCR-PGQFRCA-N---GP-----CIPQAWKCDVDNDCGDH--SDE-PIEE--CMS--- |
| megalin_ldra_2 | -VTCQ-QGYFKCQ-SE--GQ-----CIPSSWVCDQDQDCDDG--SDE--RQD--CSQ--- |
| megalin_ldra_21 | -RTCL-ADEFKCD-G---GR-----CIPSEWICDGDNDCGDM--SDEDKRHQ--CQN--- |
| LRP8_ldlra_1_v2 | -KDCE-KDQFQCR-N---ER-----CIPSVWRCDEDDDCLDH--SDE---DD--CPK--- |
| LRP8_ldlra_1 | -KECE-KDQFQCR-N---ER-----CIPSVWRCDEDDDCLDH--SDE---DD--CPK--- |
| megalin_ldra_18 | -RDCNATTEFMCN-N---RF-----CIPREFICNGVDNCHDNNTSDE---KN--CPD--- |
| megalin_ldra_30 | --------EFSCKTN---YR-----CIPKWAVCNGVDDCRDN--SDE---QG--CEE--- |
| megalin_ldra_25 | -PTCPP-HEFKCD-N---GR-----CIEMMKLCNHLDDCLDN--SDE---KG--CGI--- |
| megalin_ldra_35 | -VPCNSPNRFRCD-N---NR-----CIYSHEVCNGVDDCGDGTDETE---EH--CRK--- |
| megalin_ldra_6 | -PTCGG-YQFTCP-S---GP-----CIYQNWVCDGEDDCKDN--GDE---DG--CES--- |
| megalin_ldra_11 | -ETCQP-SQFNCP-N---HR-----CIDLSFVCDGDKDCVDG--SDE---VG--CV---- |
| LRP8_ldlra_5 | ---CAP-HEFQCG-N---RS-----CLAAVFVCDGDDDCGDG--SDE---RG--CAD--- |
| megalin_ldra_8 | -EQCGL-FSFPCK-N---GR-----CVPNYYLCDGVDDCHDN--SDE---QL--CGT--- |
| megalin_ldra_34 | -AYCQA-TMFECK-N---NV-----CIPPYWKCDGDDDCGDG--SDEE-LHL--CLD--- |
| megalin_ldra_17 | -HTCS-PTAFTCA-N---GR-----CVQYSYRCDYYNDCGDG--SDE---AG--CLF--- |
| megalin_ldra_23 | -RTCS-ENEFTCG-Y---GL-----CIPKIFRCDRHNDCGDY--SDE---RG--CLY--- |
| megalin_ldra_3 | -STCS-SHQITCS-N---GQ-----CIPSEYRCDHVRDCPDG--ADE---ND--CQY--- |
| megalin_ldra_9 | -NTCS-SSAFTCG-H---GE-----CIPAHWRCDKRNDCVDG--SDE---HN--CPT--- |
| megalin_ldra_14 | -KTCP-SSYFHCD-N---GN-----CIHRAWLCDRDNDCGDM--SDE---KD--CPT--- |
| megalin_ldra_10 | -ASCL-DTQYTCD-N---HQ-----CISKNWVCDTDNDCGDG--SDE---KN--CNS--- |
| megalin_ldra_24 | -QTCQ-QNQFTCQ-N---GR-----CISKTFVCDEDNDCGDG--SDEL-MHL--CHT--- |
| megalin_ldra_4 | -PTC---EQLTCD-N---GA-----CYNTSQKCDWKVDCRDS--SDE---IN--CTE--- |
| megalin_ldra_16 | -ERCG-ASSFTCS-N---GR-----CISEEWKCDNDNDCGDG--SDEM-ESV--CAL--- |
| LRP8_ldlra_3 | -KTCA-DSDFTCD-N---GH-----CIHERWKCDGEEECPDG--SDES-EAT--CTK--- |
| megalin_ldra_1 | -QECD-SAHFRCG-S---GH-----CIPADWRCDGTKDCSDD--ADE---IG--CAV--- |
| LRP8_ldlra_4 | --------------S---NK-----CVPASWRCDGEKDCEGG--ADE---AG--CAT--- |
| megalin_ldra_12 | -LNCT-ASQFKCA-S---GDK----CIGVTNRCDGVFDCSDN--SDE---AG--CPT--- |
| LRP8_ldlra_7 | AACAT-VSQFACR-S---GE-----CVHLGWRCDGDRDCKDK--SDE---AD--CPL--- |
| megalin_ldra_33 | -RTCH-PEYFQCT-S---GH-----CVHSELKCDGSADCLDA--SDE---AD--CPT--- |
| megalin_ldra_27 | -RFCR-LGQFQCS-D---GN-----CTSPQTLCNAHQNCPDG--SDE---DR--LLCEN- |
| LRP8_ldlra_2 | -GTCR-GDEFQCG-D---GT-----CVLAIKHCNQEQDCPDG--SDE---AG--CLQ--- |
| megalin_ldra_7 | -HKCS-PREWSCP-E---SG----RCISIYKVCDGILDCPGR--EDEN--NT--STGKYC |
| megalin_ldlra_15 | -FRCP-SWQWQCL-G---HN----ICVNLSVVCDGIFDCPNG--TDES--PL--CNG--- |
| megalin_ldra_13 | -GMCH-SDEFQCQ-E---DG----ICIPNFWECDGHPDCLYG--SDEH--NA--CVP--- |
| megalin_ldra_26 | -PMCS-STQFLCA-N---NE----KCIPIWWKCDGQKLCSDG--SDEL--AL--CPQ--- |
| megalin_ldra_22 | -QNCS-DSEFLCV-N---DRPPDRRCIPQSWVCDGDVDCTDG--YDEN--QN--CTR--- |
| megalin_ldra_20 | -HTCS-SSEFQCA-S---GR----CIPQHWYCDQETDCFDA--SDEP--AS--CGH--- |
| megalin_ldra_28 | -HHCD-SNEWQCA-N---KR----CIPESWQCDTFNDCEDN--SDED--SSH-CAS--- |
| megalin_ldlra_5 | -- |
| megalin_ldlra_36 | -- |
| LRP8_ldlra_6 | -- |
| LRP4_ldlra | -- |
| megalin_ldlra_19 | -- |
| megalin_ldlra_31 | -- |
| megalin_ldlra_32 | -- |
| megalin_ldra_29 | -- |
| megalin_ldlra_2 | -- |
| megalin_ldlra_21 | -- |
| LRP8_ldlra_1_v2 | -- |
| LRP8_ldlra_1 | -- |
| megalin_ldra_18 | -- |
| megalin_ldra_30 | -- |
| megalin_ldlra_25 | -- |
| megalin_ldra_35 | -- |
| megalin_ldra_6 | -- |
| megalin_ldra_11 | -- |
| LRP8_ldlra_5 | -- |
| megalin_ldlra_8 | -- |
| megalin_ldra_34 | -- |
| megalin_ldra_17 | -- |
| megalin_ldlra_23 | -- |
| megalin_ldra_3 | -- |
| megalin_ldlra_9 | -- |
| megalin_ldlra_14 | -- |
| megalin_idira_10 | -- |
| megalin_ldlra_24 | -- |
| megalin_ldlra_4 | -- |
| megalin_ldra_16 | -- |
| LRP8_ldlra_3 | -- |
| megalin_ldra_1 | -- |
| LRP8_ldlra_4 | -- |
| megalin_ldlra_12 | -- |
| LRP8_ldlra_7 | -- |
| megalin_ldra_33 | -- |
| megalin_ldra_27 | -- |
| LRP8_ldlra_2 | -- |
| megalin_ldra_7 | SM |
| megalin_ldlra_15 | -- |
| megalin_ldra_13 | -- |
| megalin_ldlra_26 | -- |
| megalin_ldra_22 | -- |
| megalin_ldlra_20 | -- |
| megalin_ldlra_28 | -- |

TABLE 5
CLUSTAL SA1 (1.8) multiple sequence alignment

| | |
|---|---|
| LRP4_ldlrb9 | ERIYWTDWQ-TK----SIQSADRLTGLD----RETL----QEN-----LENLMDIHVFHR |
| LRP8_ldlrb_5 | DKVFWTDLE-NE----AIFSANRLNGLE----ISIL----AEN-----LNNPHDIVIFHE |
| megalin_ldlrb_19 | DTIYWTDWN-TR----TVEKGNKYDGSN----RQTL----VNT-----THRPFDIHVYHP |
| megalin_ldlrb22 | DRIYWSDFK-ED----VIET-IKYDGTD----RRVI----AKE-----A----------- |
| LRP4_ldlrb2 | DKLYWTDSG-TS----RIEV-ANLDGAH----RKVL----LWQN----LEKPRAIALHPM |
| LRP4_ldlrb11 | RKVYWTDTG-TN----RIEV-GNLDGSM----RKVL----VWQN----LDSPRAIVLYHE |
| LRP4_ldlrb6 | NKLYWTDAG-TD----RIEV-ANTDGSM----RTVL----IWEN----LDRPRDIVVEPM |
| LRP4_ldlrb15 | RNLYWTDTG-RN----TIEA-SRLDGSC----RKVL----INNS----LDEPRAIAVFPR |
| megalin_ldlrb_5 | KNLYWTDSH-YK----SISV-MRLADKT----RRTV----VQY-----LNNPRSVVVHPF |
| LRP8__ldlrb_2 | KHIYWTDSG-NK----TISV-ATVDGGR----RRTL----FSRN----LSEPRAIAVDPL |
| megalin_ldlrb_20 | RHIYWSDVK-NK----RIEV-AKLDGRY----RKWL----ISTD----LDQPAAIAVNPK |
| megalin_ldlrb_2 | NKIYLVETK-VN----RIDM-VNLDGSY----RVTL----ITEN----LGHPRGIAVDPT |
| megalin_ldlrb_8 | RNLYWTDYA-LE----TIEV-SKIDGSH----RTVL----ISKN----LTNPRGLALDPR |
| megalin_ldlrb_16 | RKLYWLDAR-LD----GLFV-SDLNGGH----RRMLAQHCVDANNTFCFDNPRGLALHPQ |
| megalin_ldlrb_11 | QKLYWAVTG-RG----VIER-GNVDGTD----RMIL----VHQ-----LSHPWGIAVH-- |
| megalin_ldlrb_12 | RYLFWADYGQRP----KIER-SFLDCTN----RTVL----VSEG----IVTPRGLAVDRS |
| LRP4_ldlrb4 | RRMYWVDAK-HH----VIER-ANLDGSH----RKAV----ISQG----LPHPFAITVFE- |
| LRP8_ldlrb_4 | QRLYWVDSK-LH----QLSS-IDFSGGN----RKTL----ISSTD---FLSHPFGIAVFE- |
| LRP4_ldlrb13 | SQLLWADAH-TE----RIEA-ADLNGAN----RNTL----VS-P----VQHPYGLTL-- |
| LRP4_ldlrb17 | RRIYWVDAH-LD----RIES-ADLNGKL----RQVL----VG-H----VSHPFALT--- |
| megalin_ldlrb_14 | DLLYWVDAS-LQ----RIER-STLTGVD----REVI----VNAA----V-HAFGLTLY-- |
| megalin_ldlrb_18 | DLLYWADAH-LG----YIEY-SDLEGHH----RHTV----YDGA----LPHPFAITIFE- |
| LRP4_ldlrb8 | QRLYWADAG-MK----TIEF-AGLDGSK----RKVL----IGSQ----LPHPFGLTLY-- |
| LRP4_ldlrb3 | GTIYWTDWGNTP----RIEA-SSMDGSG----RRII----ADTH----LFWPNGLTIDYA |
| megalin_ldlrb_9 | HLLFWSDWGHHP----RIER-ASMDGSM----RTVI----VQDK----IFWPCGLTIDYP |
| megalin_ldlrb_21 | GLMFWTDWGKEP----KIES-AWMNGED----RNIL----VFED----LGWPTGLSIDYL |
| LRP4_ldlrb7 | GYMYWTDWGASP----KIER-AGMDASG----RQVI----ISSN----LTWPNGLAIDYG |
| LRP4_ldlrb12 | GFMYWTDWGENA----KLER-SGMDGSD----RAVL----INNN----LGWPNGLTVDKA |
| LRP8_ldlrb_3 | GFMYWSDWGDQA----KIEK-SGLNGVD----RQTL----VSDN----IEWPNGITDLDLL |
| LRP4_ldlrb16 | GYLFWTDWGHIA----KIER-ANLDGSE----RXVL----INTD----LGWPNGLTLDYD |
| megalin_ldlrb_6 | GYLFFTDWFRPA----KIMR-AWSDGSH----LLPV----INTT----LGWPNGLAIDWA |
| megalin_ldlrb_3 | GYLFFSDWESLSGE-PKLER-AFMDGSN----RKDL----VKTK----LGWPAGVTLDMI |
| megalin_ldlrb_13 | GYLYWADWDTHA----KIER-ATLGGNF----RVPI----VNSS----LVMPSGLTLDYE |
| megalin_ldlrb_17 | GYLYWADWGHRA----YIGR-VGMDGTN----KSVI----ISTK----LEWPNGITIDYT |
| megalin_ldlrb_10 | GKLYWSDQGTDSGVPAKIAS-ANMDGTS----VKTL----FTGN----LEHLECVTLDIE |
| LRP4_ldlrb_10 | GKVYWSDST-LH----RISR-ANLDGSQ----HEDI----ITTG----LQTTDGLAVDAI |
| LRP4_ldlrb14 | GKVYYTDVF-LD----VIRR-ADLNGSN----METV----IGRG----LKTTDGLAVDWV |
| LRP4_ldlrb1 | ELVFWSDVT-LD----RILR-ANLNGSN----VEEV----VSTG----LESPGGLAVDWV |
| LRP8_ldlrb_1 | NRIYWCDLS-YR----KIYS-AYMDKASDPKEQEVL----IDEQ----LHSPEGLAVDWV |
| LRP8ldlrb_1_v2 | NRIYWCDLS-YR----KIYS-AYMDKASDPKEREVL----IDEQ----LHSPEGLAVDWV |
| LRP4ldlrb5 | DHVYWTDVS-TD----TISR-AKWDGTG---VQEVV----VDTS----LESPAGLAIDWV |
| megalin_ldlrb_1 | QRVFWTDTV-QN----KVFS-VDINGLN---IQEV-----LNVS----VETPENLAVDWV |
| megalin_ldlrb_7 | GRIFWSDAT-QG----KTWS-AFQNGTD---RRVV-----FDSS----IILTETIAIDWV |
| megalin_ldlrb_4 | STIFFSDMS-KH----MIFK-QKIDGTG---REIL-----AANR----VENVESLAFDWI |
| megalin_ldlrb_15 | KRLYWIDTQ-RQ----VIER-MFLNKTN---KETI-----INHR----LPAAESLAVDWV |

TABLE 6
CLUSTAL W (1.8) multiple sequence alignment

| | |
|---|---|
| 3449306_3449305_EGF2 | CAMENGGCSHLCLRSPNPSGFSCTCPTGINLLSDGKTC |
| 3449306_3449305_EGF3 | CGSRNGGCSHLCL--PRPSGFSCACPTGIQLKGDGKTC |
| 3449306_3449305_EGF1 | CNVNNGGCAQKCQ--MVRGAVQCTCHTGYRLTEDGHTC |
| | * .****:: * ....*:* ** .* **:** |

TABLE 7
CLUSTAL W (1.8) multiple sequence alignment

| | |
|---|---|
| 3449306_3449305_ldlrb1 | ELVFWSDV-TLDRILRAN-LNGSNVEEVVSTGLESPGGLAVDWV |
| 3449306_3449305_ldlrb14 | GKVYYTDV-FLDVIRRAD-LNGSNMETVIGRGLKTTDGLAVDWV |
| 3449306_3449305_ldlrb10 | GKVYWSDS-TLHRISRAN-LDGSQHEDIITTGLQTTDGLAVADI |
| 3449306_3449305_ldlrb5 | DHVYWTDV-STDTISRAK-WDGTGQEVVVDTSLESPAGLAIDWV |
| 3449306_3449305_ldlrb3 | GTIYWTDWGNTPRIEASS-MDGSGRRIIADTHLFWPNGLTIDYA |
| 3449306_3449305_ldlrb7 | GYMYWTDWGASPKIERAG-MDASGRQVIISSNLTWPNGLAIDYG |
| 3449306_3449305_ldlrb12 | GFMYWTDWGENAKLERSG-MDGSDRAVLINNNLGWPNGLTVDKA |
| 3449306_3449305_ldlrb16 | GYLFWTDWGHIAKIERAN-LDGSERKVLINTDLGWPNGLTLDYD |
| 3449306_3449305_ldlrb2 | DKLYWTDS-GTSRIEVAN-LDGAHRKVLLWQNLEKPRAIALHPM |
| 3449306_3449305_ldlrb11 | RKVYWTDT-GTNRIEVGN-LDGSMRKLVLWQNLDSPRAIVLYHE |
| 3449306_3449305_ldlrb6 | NKLYWTDA-GTDRIEVAN-TDGSMRTVLIWENLDRPRDIVVEPM |
| 3449306_3449305_ldlrb15 | RNLYWTDT-GRNTIEASR-LDGSCRKVLINNSLDEPRAIAVFPR |
| 3449306_3449305_ldlrb13 | SQLLWADA-HTERIEAAD-LNGANRHTLVS-PVQHPYGLTL--- |
| 3449306_3449305_ldlrb17 | RRIYWVDA-HLDRIESAD-LNGKLRQVLVG-HVSHPFALT---- |
| 3449306_3449305_ldlrb8 | QRLYWADA-GMKTIEFAG-LDGSKRKVLIGSQLPHPFGLTLY-- |
| 3449306_3449305_ldlrb4 | RRMYWVDA-KHHVIERAN-LDGSHRKAVISQGLPHPFAITVFE- |
| 3449306_3449305_ldlrb9 | ERIYWTDW-QTKSIQSADRLTGLDRETLQE-NLENLMDIHVFHR |
| | : : * : . . : : : |

TABLE 8
CLUSTAL W (1.8) multiple sequence alignment

| | |
|---|---|
| megalin_{—} | CDILG-SCSQHCYNMRGSFR--CSCDTGYMLESDGRTC |
| EGF_2 | |
| megalin_{—} | CTEMPFVCSQKCENVIGSYI--CKCAPGYLREPDGKTC |
| EGF_4 | |
| megalin_{—} | CLENNGGCSHLCFALPGLHTPKCDCAFGTLQ-SDGKNC |
| EGF_3 | |
| megalin_{—} | CSDFNGGCTHECVQEP--FGAKCLCPLGFLLANDSKTC |
| EGF_1 | |
| megalin_{—} | CMH-GGNCYFDETDLP---K--CKCPSGYT----GKYC |
| EGF_5 | * * * * * .: * |

TABLE 9
CLUSTAL W (1.8) multiple sequence alignment

| | |
|---|---|
| megalin_ldlra_1 | -QECDSA-HFRCG-SG----HCIPADWRCDGTKDCSDD--ADE---IG-CAV----- |
| megalin_ldlra_12 | -LNCTAS-QFKCA-SG---DKCIGVTNRCDGVFDCSDN--SDE---AG-CPT----- |
| megalin_ldlra_33 | -RTCHPE-YFQCT-SG----HCVHSELKCDGSADCLDA--SDE---AD-CPT----- |
| megalin_ldlra_4 | -PTC--E-QLTCD-NG----ACYNTSQKCDWKVDCRDS--SDE---IN-CTE----- |
| megalin_ldlra_27 | -RFCRLG-QFQCS-DG----NCTSPQTLCNAHQNCPDG--SDED-RLL-CEN----- |
| megalin_ldlra_6 | -PTCGGY-QFTCP-SG----RCIYQNWVCDGEDDCKDN--GDE---DG-CES----- |
| megalin_ldlra_11 | -ETCQPS-QFNCP-NH----RCIDLSFVCDGDKDCVDG--SDE---VG-CV------ |
| megalin_ldlra_18 | RDCNATT-EFMCN-NR----RCIPREFICNGVDNCHDNNTSDE---KN-CPD----- |
| megalin_ldlra_30 | --------EFSCKTNY----RCIPKWAVCNGVDDCRDN--SDE---QG-CDD----- |
| megalin_ldlra_35 | VPCNSPN-RFRCD-NN----RCIYSHEVCNGVDDCGDGTDETE---EH-CRK----- |
| megalin_ldlra_25 | -PTCPPH-EFKCD-NG----RCIEMMKLCNHLDDCLDN--SDE---KG-CGI----- |
| megalin_ldlra_8 | -EQCGLF-SFPCK-NG----RCVPNYYLCDGVDDCHDN--SDE---GL-CGT----- |
| megalin_ldlra_2 | -VTCQQG-YFKCQ-SE---GQCIPSSWVCDQDQDCDDG--SDE--RQD-CSQ----- |
| megalin_ldlra_21 | -RTCLAD-EFKCD-G----GRCIPSEWICDGDNDCGDM--SDEDKRHQ-CQN----- |
| megalin_ldlra_14 | -KTCPSS-YFHCD-NG---N-CIHRAWLCDRDNDCGDM--SDE-KD---CPT----- |
| megalin_ldlra_19 | -RTCQSG-YTKCH-NS---NICIPRVYLCDGDNDCGDN--SDE-NPTY-CTT----- |
| megalin_ldlra_31 | -RTCHPVGDFRCK-N----HHCIPLRWQCDGQNDCGDN--SDE---EN-CAP----- |
| megalin_ldlra_32 | -RECTES-EFRCV-N----QQCIPSRWICDHYNDCGDN--SDE---RD-CEM----- |
| megalin_ldlra_29 | -RTCRPG-QFRCA-N----GRCIPQAWKCDVDNDCGDH--SDEP-IEE-CMS----- |
| megalin_ldlra_10 | -ASCLDT-QYTCD-N----HQCISKNWVCDTDNDCGDG--SDE---KN-CNS----- |
| megalin_ldlra_24 | -QTCQQN-QFTCQ-N----GRCISKTFVCDEDNDCGDG--SDEL-MHL-CHT----- |
| megalin_ldlra_34 | -AYCQAT-MFECK-N----HVCIPPYWKCDGDDDCGDG--SDEE-LHL-CLD----- |
| megalin_ldlra_16 | -ERCGAS-SFTCS-N----GRCISEEWKCDNDNDCGDG--SDEM-ESV-CAL----- |
| megalin_ldlra_17 | -HTCSPT-AFTCA-N----GRCVQYSYRCDYYNDCGDG--SDE---AG-CLF----- |
| megalin_ldlra_23 | -RTCSEN-EFTCG-Y----GLCIPKIFRCDRHNDCGDY--SDE---RG-CLY----- |
| megalin_ldlra_9 | -NTCSSS-AFTCG-H----GECIPAHWRCDKRNDCVDG--SDE---HN-CPT----- |
| megalin_ldlra_3 | -STCSSH-QITCS-N----GQCIPSEYRCDHVRDCPDG--ADE---ND-CQY----- |
| megalin_ldlra_ | ---CLHN-EFSCG-N----GECIPRAYVCDHDNDCQDG--SDE---HA-CNY----- |
| megalin_ldlra_36 | -KPCTEY-EYKCG-N----GHCIPHDNVCDDADDCGDW--SDE---LG-CNK----- |
| megalin_ldlra_7 | -HKCSPR-EWSCPESG----RCISIYKVCDGILDCPGR--EDE--NNT-STGKYCSM |
| megalin_ldlra_15 | -FRCPSW-QWQCLGHN----ICVNLSVVCDGIFDCPNG--TDE--SPL-CNG----- |
| megalin_ldlra_13 | -GMCHSD-EFQCQEDG----ICIPNFWECDGHPDCLYG--SDE--HNA-CVP----- |
| megalin_ldlra_26 | -PMCSST-QFLCANNE----KCIPIWWKCDGQKDCSDG--SDE--LAL-CPQ----- |
| megalin_ldlra_22 | -QNCSDS-EFLCVNDRPPDRRCIPQSWVCDGDVDCTDG--YDE--NQN-CTR----- |
| megalin_ldlra_20 | -HTCSSS-EFQCASGR-----CIPQHWYCDQETDCFDA--SDE--PAS-CGH----- |
| megalin_ldlra_28 | -HHCDSN-EWQCANKR-----CIPESWQCDTFNDCEDN--SDE--DSSHCAS----- |
| | * * *: :* * . |

TABLE 10
CLUSTAL W (1.8) multiple sequence alignment

| | |
|---|---|
| megalin_ldlrb_19 | DTIYWTDWNTR----T-VEKGNKYDGSNRQTLVNTTH---------RPFDIHVYHP |
| megalin_ldlrb_22 | DRIYWSDFKED----V-IET-IKYDGTDRRVIAKEA-------------------- |
| megalin_ldlrb_14 | DLLYWVDASLQ----R-IER-STLTGVDREVIVNAAV---------NAFGLTLY-- |
| megalin_ldlrb_18 | DLLYWADAHLG----Y-IEY-SDLEGHHRHTVYDGALP--------HPFAITIFE- |
| megalin_ldlrb_11 | QKLYWAVTGRG----V-IER-GNVDGTDRMILVHQLS---------HPWGIAVH-- |
| megalin_ldlrb_2 | NKIYLVETKVN----R-IDM-VNLDGSYRVTLITEN--------LGHPRGIAVDPT |
| megalin_ldlrb_20 | RHIYWSDVKNK----R-IEV-AKLDGRYRKWLISTD--------LDQPAAIAVNPK |
| megalin_ldlrb_5 | KNLYWTDSHYK----S-ISV-MRLADKTRRTVVQY---------LNNPRSVVVHPF |
| megalin_ldlrb_16 | RKLYWLDARLD----G-LFV-SDLNGGHRRMLAQHCVDANNTFCFDNPRGLALHPQ |
| megalin_ldlrb_8 | RNLYWTDYALE----T-IEV-SKIDGSHRTVLISKN--------LTNPRGLALDPR |
| megalin_ldlrb_12 | RYLFWADYGQR----PKIER-SFLDCTNRTVLVSEG--------IVTPRGLAVDRS |
| megalin_ldlrb_3 | GLYFFSDWESLS-GEPKLER-AFMDGSNRKDLVKTK--------LGWPAGVTLDMI |
| megalin_ldlrb_6 | GYLFFTDWFRP----AKIMR-AWSDGSHLLPVINTT--------LGWPNGLAIDWA |
| megalin_ldlrb_13 | GYLYWADWDTH----AKIER-ATLGGNFRVPIVNSS--------LVMPSGLTLDYE |
| megalin_ldlrb_9 | HLLFWSDWGHH----PRIER-ASMDGSMRTVIVQDK--------IFWPCGLTIDYP |
| megalin_ldlrb_21 | GLMFWTDWGKE----PKIES-AWMNGEDRNILVFED--------LGWPTGLSIDYL |
| megalin_ldlrb_17 | GYLYWADWGHR----AYIGR-VGMDGTNKSVIISTK--------LEWPNGITIDYT |
| megalin_ldlrb_10 | GKLYWSDQGTDSGVPAKIAS-ANMDGTSVKTLFTGN--------LEHLECVTLDIE |
| megalin_ldlrb_1 | QRVFWTDTVQN----K-VFS-VDINGLNIQEVLNVS--------VETPENLAVDWV |
| megalin_ldlrb_7 | GRIFWSDATQG----K-TWS-AFQNGTDRRVVFDSS--------IILTETIAIDWV |
| megalin_ldlrb_15 | KRLYWIDTQRQ----V-IER-MFLNKTNKETIINHR--------LPAAESLAVDWV |
| megalin_ldlrb_4 | STIFFSDMSKH----M-IFK-QKIDGTGREILAANR--------VENVESLAFDWI |
| | :: : |

TABLE 11
CLUSTAL W (1.8) multiple sequence alignment

| | |
|---|---|
| LRP8_ldlra_2 | GTCRG-DEFQCGD---GTCVLAIKHCNQEQDCPDGSDEA--GCLQ |
| LRP8_ldlra_5 | --CAP-HEFQCGN---RSCLAAVFVCDGDDDCGDGSDER--GCAD |
| LRP8_ldlra_3 | KTCAD-SDFTCDN---GHCIHERWKCDGEEECPDGSDESEATCTK |
| LRP8_1dlra_6 | PACGP-REFRCGGDGGGACIPERWVCDRQFDCEDRSDEAAELCGR |
| LRP8_ldlra_1_v2 | RDCEK-DQFQCRN---ERCIPSVWRCDEDDDCLDHSDED--DCPK |
| LRP8_ldlra_1 | KECEK-DQFQCRN---ERCIPSVWRCDEDDDCLDHSDED--DCPK |
| LRP8_ldlra_7 | AACATVSQFACRS---GECVHLGWRCDGDRDCKDKSDEA--DCPL |
| LRP8_ldlra_4 | ------------S---HKCVPASWRCDGEKDCEGGADEA--GCAT |
| | . *: *: : :* . :** * |

TABLE 12
CLUSTAL W (1.8) multiple sequence alignment

| | |
|---|---|
| LRP8_ldlrb_2 | KHIYWTDSGNKT-ISVATVDGG----RRRTLFSRN--LSEPRAIAVDPL |
| LPR8_ldlrb_3 | GFMYWSDWGDQAKIEKSGLNGV----DRQTLVSDN--IEWPNGITLDLL |
| LRP8_ldlrb_1 | NRIYWCDLSYRK-IYSAYMDKASDPKEQEVLIDEQ--LHSPEGLAVDWV |
| LRP8_ldlrb_1_v2 | NRIYWCDLSYRK-IYSAYMDKASDPKEREVLIDEQ--LHSPEGLAVDWV |
| LRP8_ldlrb_5 | DKVFWTDLENEAIFSANRLNGL-----EISILAEN--LNNPHDIVIFHE |
| LRP8_ldlrb_5 | QRLYWVDSKLHQ-LSSIDFSGG----NRKTLISSTDFLSHPFGIAVFE- |
| | ::* * . : .. . :. : * :.: |

TABLE 13

| | | | |
|---|---|---|---|
| Sequence ID | Pfam | Start | Stop |

| | | | |
|---|---|---|---|
| 22065231:22065230 | EGF | 943 | 978 |
| 22065231:22065230 | EGF | 636 | 673 |
| 22065231:22065230 | ldl_recept_b | 809 | 851 |
| 22065231:22065230 | ldl_recept_b | 1113 | 1155 |
| 22065231:22065230 | ldl_recept_b | 153 | 194 |
| 22065231:22065230 | ldl_recept_b | 501 | 543 |
| 22065231:22065230 | ldl_recept_b | 196 | 238 |
| 22065231:22065230 | ldl_recept_b | 240 | 280 |
| 22065231:22065230 | ldl_recept_b | 766 | 807 |
| 22065231:22065230 | ldl_recept_b | 415 | 456 |
| 22065231:22065230 | ldl_recept_b | 1070 | 1111 |
| 22065231:22065230 | ldl_recept_b | 458 | 499 |
| 22065231:22065230 | ldl_recept_b | 723 | 764 |
| 22065231:22065230 | ldl_recept_b | 110 | 151 |
| 22065231:22065230 | ldl_recept_b | 545 | 584 |
| 22065231:22065230 | ldl_recept_b | 1027 | 1068 |
| 22065231:22065230 | ldl_recept_b | 586 | 627 |
| 22065231:22065230 | ldl_recept_b | 1157 | 1193 |
| 22065231:22065230 | ldl_recept_b | 853 | 890 |
| 3449306:3449305 | EFG | 984 | 1019 |
| 3449306:3449305 | EFG | 677 | 714 |
| 3449306:3449305 | EFG | 29 | 64 |
| 3449306:3449305 | ldl_recept_a | 1 | 22 |
| 3449306:3449305 | ldl_recept_b | 850 | 892 |
| 3449306:3449305 | ldl_recept_b | 1154 | 1196 |
| 3449306:3449305 | ldl_recept_b | 194 | 235 |
| 3449306:3449305 | ldl_recept_b | 542 | 584 |
| 3449306:3449305 | ldl_recept_b | 237 | 279 |
| 3449306:3449305 | ldl_recept_b | 281 | 321 |
| 3449306:3449305 | ldl_recept_b | 807 | 848 |
| 3449306:3449305 | ldl_recept_b | 456 | 497 |
| 3449306:3449305 | ldl_recept_b | 1111 | 1152 |
| 3449306:3449305 | ldl_recept_b | 499 | 540 |
| 3449306:3449305 | ldl_recept_b | 764 | 805 |
| 3449306:3449305 | ldl_recept_b | 151 | 192 |
| 3449306:3449305 | ldl_recept_b | 586 | 625 |
| 3449306:3449305 | ldl_recept_b | 1068 | 1109 |
| 3449306:3449305 | ldl_recept_b | 627 | 668 |
| 3449306:3449305 | ldl_recept_b | 1198 | 1234 |
| 3449306:3449305 | ldl_recept_b | 894 | 931 |

TABLE 14

| | | | |
|---|---|---|---|
| Sequence ID | Pfam | Start | Stop |

| | | | |
|---|---|---|---|
| NP_004516:NM_004525 | EGF | 3156 | 3191 |
| NP_004516:NM_004525 | EGF | 1394 | 1428 |
| NP_004516:NM_004525 | EGF | 1353 | 1388 |
| NP_004516:NM_004525 | EGF | 2346 | 2382 |
| NP_004516:NM_004525 | EGF | 4383 | 4410 |
| NP_004516:NM_004525 | ldl_recept_a | 3633 | 3673 |
| NP_004516:NM_004525 | ldl_recept_a | 3717 | 3756 |
| NP_004516:NM_004525 | ldl_recept_a | 2862 | 2901 |
| NP_004516:NM_004525 | ldl_recept_a | 1065 | 1103 |
| NP_004516:NM_004525 | ldl_recept_a | 1269 | 1307 |
| NP_004516:NM_004525 | ldl_recept_a | 3073 | 3111 |
| NP_004516:NM_004525 | ldl_recept_a | 1147 | 1184 |
| NP_004516:NM_004525 | ldl_recept_a | 2739 | 2777 |
| NP_004516:NM_004525 | ldl_recept_a | 3030 | 3070 |
| NP_004516:NM_004525 | ldl_recept_a | 65 | 105 |
| NP_004516:NM_004525 | Idl_recept_a | 2991 | 3029 |
| NP_004516:NM_004525 | ldl_recept_a | 3796 | 3834 |
| NP_004516:NM_004525 | ldl_recept_a | 2904 | 2945 |
| NP_004516:NM_004525 | ldl_recept_a | 1024 | 1062 |
| NP_004516:NM_004525 | ldl_recept_a | 3840 | 3880 |
| NP_004516:NM_004525 | ldl_recept_a | 2698 | 2738 |
| NP_004516:NM_004525 | ldl_recept_a | 1107 | 1145 |
| NP_004516:NM_004525 | ldl_recept_a | 220 | 258 |
| NP_004516:NM_004525 | ldl_recept_a | 3592 | 3632 |
| NP_004516:NM_004525 | ldl_recept_a | 26 | 64 |
| NP_004516:NM_004525 | ldl_recept_a | 3510 | 3550 |
| NP_004516:NM_004525 | ldl_recept_a | 3757 | 3795 |
| NP_004516:NM_004525 | ldl_recept_a | 106 | 144 |
| NP_004516:NM_004525 | ldl_recept_a | 3881 | 3922 |
| NP_004516:NM_004525 | ldl_recept_a | 183 | 219 |
| NP_004516:NM_004525 | ldl_recept_a | 2946 | 2990 |
| NP_004516:NM_004525 | ldl_recept_a | 2820 | 2861 |
| NP_004516:NM_004525 | ldl_recept_a | 3926 | 3964 |
| NP_004516:NM_004525 | ldl_recept_a | 1228 | 1268 |
| NP_004516:NM_004525 | ldl_recept_a | 1185 | 1224 |
| NP_004516:NM_004525 | ldl_recept_a | 3683 | 3716 |
| NP_004516:NM_004525 | ldl_recept_a | 2778 | 2819 |
| NP_004516:NM_004525 | ldl_recept_a | 264 | 309 |
| NP_004516:NM_004525 | ldl_recept_a | 145 | 181 |
| NP_004516:NM_004525 | ldl_recept_a | 3551 | 3591 |
| NP_004516:NM_004525 | ldl_recept_a | 1310 | 1350 |
| NP_004516:NM_004525 | ldl_recept_b | 3333 | 3375 |
| NP_004516:NM_004525 | ldl_recept_b | 1521 | 1562 |
| NP_004516:NM_004525 | ldl_recept_b | 3282 | 3331 |
| NP_004516:NM_004525 | ldl_recept_b | 4197 | 4239 |
| NP_004516:NM_004525 | ldl_recept_b | 1883 | 1929 |
| NP_004516:NM_004525 | ldl_recept_b | 1566 | 1608 |
| NP_004516:NM_004525 | ldl_recept_b | 4154 | 4195 |
| NP_004516:NM_004525 | ldl_recept_b | 838 | 880 |
| NP_004516:NM_004525 | ldl_recept_b | 479 | 520 |
| NP_004516:NM_004525 | ldl_recept_b | 2519 | 2561 |
| NP_004516:NM_004525 | ldl_recept_b | 3377 | 3417 |
| NP_004516:NM_004525 | ldl_recept_b | 436 | 477 |
| NP_004516:NM_004525 | ldl_recept_b | 2202 | 2244 |
| NP_004516:NM_004525 | ldl_recept_b | 522 | 567 |
| NP_004516:NM_004525 | ldl_recept_b | 796 | 836 |
| NP_004516:NM_004525 | ldl_recept_b | 2563 | 2601 |
| NP_004516:NM_004525 | ldl_recept_b | 1478 | 1519 |
| NP_004516:NM_004525 | ldl_recept_b | 3239 | 3280 |
| NP_004516:NM_004525 | ldl_recept_b | 3418 | 3459 |
| NP_004516:NM_004525 | ldl_recept_b | 1931 | 1969 |
| NP_004516:NM_004525 | ldl_recept_b | 753 | 794 |
| NP_004516:NM_004525 | ldl_recept_b | 4242 | 4271 |

TABLE 15

| | | | |
|---|---|---|---|
| Sequence ID | Pfam | Start | Stop |

| | | | |
|---|---|---|---|
| NP_059992:NM_017522 | ldl_recept_a | 84 | 124 |
| NP_059992:NM_017522 | ldl_recept_a | 45 | 83 |
| NP_059992:NM_017522 | ldl_recept_a | 168 | 206 |
| NP_059992:NM_017522 | ldl_recept_a | 138 | 165 |
| NP_059992:NM_017522 | ldl_recept_b | 423 | 465 |
| NP_059992:NM_017522 | ldl_recept_b | 380 | 421 |
| NP_059992:NM_017522 | ldl_recept_b | 333 | 378 |
| NP_059992:NM_017522 | ldl_recept_b | 467 | 509 |
| NP_059992:NM_017522 | ldl_recept_b | 510 | 551 |
| NP_150643:NM_033300 | EFG | 170 | 204 |
| NP_150643:NM_033300 | ldl_recept_a | 84 | 124 |
| NP_150643:NM_033300 | ldl_recept_a | 45 | 83 |
| NP_150643:NM_033300 | ldl_recept_a | 138 | 165 |
| NP_150643:NM_033300 | ldl_recept_b | 382 | 424 |
| NP_150643:NM_033300 | ldl_recept_b | 339 | 380 |
| NP_150643:NM_033300 | ldl_recept_b | 426 | 468 |
| NP_150643:NM_033300 | ldl_recept_b | 292 | 337 |
| NP_150643:NM_033300 | ldl_recept_b | 469 | 510 |
| NP_004622:NM_004631 | ldl_recept_a | 84 | 124 |
| NP_004622:NM_004631 | ldl_recept_a | 257 | 296 |
| NP_004622:NM_004631 | ldl_recept_a | 45 | 83 |
| NP_004622:NM_004631 | ldl_recept_a | 167 | 203 |
| NP_004622:NM_004631 | ldl_recept_a | 297 | 335 |
| NP_004622:NM_004631 | ldl_recept_a | 204 | 247 |
| NP_004622:NM_004631 | ldl_recept_a | 138 | 165 |
| NP_004622:NM_004631 | ldl_recept_b | 552 | 594 |
| NP_004622:NM_004631 | ldl_recept_b | 509 | 550 |
| NP_004622:NM_004631 | ldl_recept_b | 596 | 638 |
| NP_004622:NM_004631 | ldl_recept_b | 462 | 507 |
| NP_004622:NM_004631 | ldl_recept_b | 639 | 680 |
| NP_004622_mod | ldl_recept_a | 84 | 124 |
| NP_004622_mod | ldl_recept_a | 257 | 296 |
| NP_004622_mod | ldl_recept_a | 45 | 83 |
| NP_004622_mod | ldl_recept_a | 167 | 203 |
| NP_004622_mod | ldl_recept_a | 297 | 335 |
| NP_004622_mod | ldl_recept_a | 204 | 247 |
| NP_004622_mod | ldl_recept_a | 138 | 165 |
| NP_004622_mod | ldl_recept_b | 552 | 594 |
| NP_004622_mod | ldl_recept_b | 509 | 550 |
| NP_004622_mod | ldl_recept_b | 596 | 638 |
| NP_004622_mod | ldl_recept_b | 462 | 507 |
| NP_004622_mod | ldl_recept_b | 639 | 680 |
| H020C00-A_0001_A03abl_ | ldl_recept_b | 84 | 124 |
| 5pclone | | | |
| H020C00-A_0001_A03abl_ | ldl_recept_b | 45 | 83 |
| 5pclone | | | |
| PLT00006704_G09abl_3pclone | no_pfam | | |

While the present invention has been described with reference to the specific embodiments thereof, it should be understood by those skilled in the art that various changes may be made and equivalents may be substituted without departing from the true spirit and scope of the invention. In addition, many modifications can be made to adapt a particular situation, material, composition of matter, process, process step or steps, to the objective, spirit and scope of the present invention. All such modifications are intended to be within the scope of the claims appended hereto.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims. Moreover, advantages described in the body of the specification, if not included in the claims, are not per se limitations to the claimed invention.

It is to be understood that both the foregoing general description and the following detailed description are exemplary and explanatory only and are not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claims.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

It will be clear that the invention may be practiced otherwise than as particularly described in the foregoing description, claims and examples. Numerous modifications and variations of the present invention are possible in light of the above teachings.

### SEQUENCE LISTING

A sequence listing transmittal sheet and a sequence listing in paper format accompanies this application.

1. A pharmaceutical composition for treatment of a disease, wherein the composition comprises a pharmaceutically acceptable carrier or excipient and at least one modulator that binds to or interferes with the activity of LRP4, LRP8, or an active fragment of either.

2. The composition of claim 1, wherein the modulator comprises a small molecule, an RNAi molecule, an anti-sense molecule, a ribozyme, or an antibody.

3. The composition of claim 2, wherein the modulator comprises an antibody.

4. The composition of claim 3, wherein the antibody is a monoclonal antibody.

5-8. (canceled)

9. The composition of claim 1, wherein the disease is a proliferative disease.

10. (canceled)

11. The composition of claim 3, wherein the antibody specifically binds to or interferes with the activity of a sequence of any of SEQ ID NOs: 10 and SEQ ID NOs: 10-15, 17-18, 28-48, or 112-126.

12. The composition of claim 3, wherein the antibody specifically binds to or interferes with the activity of a ligand of any one LRP4, a ligand of LRP8, or an active fragment of either.

13. A method of treatment of a disease in a subject, comprising the steps of:
(a) providing a composition comprising a pharmaceutically acceptable carrier or excipient and at least one modulator that binds to or interferes with the activity of LRP2 (megalin), LRP4, LRP8, or an active fragment of either; and
(b) administering an amount of the composition to the subject effective to treat the disease.

14. The method of claim 13, wherein the disease comprises a proliferative disease.

15. (canceled)

16. The method of claim 14, wherein the proliferative disease comprises a tumor.

17. The method of claim 16, wherein the tumor is selected from splenic tumor, cervical tumor, leukemia, stomach tumor, liver tumor, thyroid tumor, skin tumor, breast tumor, lung tumor, kidney tumor, brain tumor, colorectal tumor, ovarian tumor, pancreatic tumor, and lymphoma.

18. The method of claim 13, wherein the disease comprises a degenerative disease.

19. (canceled)

20. The method of claim 18, wherein the degenerative disease comprises Alzheimer's disease.

21-45. (canceled)

46. The method of claim 13, wherein the composition comprises an antibody that specifically binds to or interferes with the activity of LRP2 (megalin) or an active fragment thereof.

47. The method of claim 46, wherein the antibody specifically binds to or interferes with a polypeptide of any of SEQ ID NOs: 16 and 49-111.

48-49. (canceled)

50. The method of claim 13, wherein the composition is administered locally or systemically.

51. The method of claim 50, wherein the composition is administered intravenously, intraperitoneally, intratumorly, intralesionally, transdermally, intrathecally, subcutaneously, intranasally or by inhalation.

52. An isolated polypeptide comprising an amino acid sequence, wherein the amino acid sequence comprises a sequence of at least six amino acid residues and wherein the amino acid sequence is any one of SEQ ID NOs: 28-126.

53. An isolated nucleic acid molecule, wherein the nucleic acid molecule comprises a polynucleotide sequence that encodes the polypeptide of claim 52.

54. A vector comprising the nucleic acid molecule of claim 53 and a regulatory sequence that regulates the expression of the nucleic acid molecule.

55. A modified cell comprising the polypeptide of claim 52, the nucleic acid molecule of claim 53, or the vector of claim 54.

56. An isolated antibody comprising an antigen-binding domain that binds to or interferes with the activity of the polypeptide of claim 52.

57-70. (canceled)

71. A composition comprising a polypeptide and a modulator, wherein the modulator specifically interferes with the activity or binding of the polypeptide, and wherein the polypeptide comprises a sequence of any of SEQ ID NOS: 10-18 and 28-126.

72. The composition of claim 71, wherein the modulator is an antibody.

73-77. (canceled)


SEQUENCE LISTING (127 sequences)

SEQ ID NO: 1-27 - skipped (27 entries)

SEQ ID NO: 28 - protein, 36 aa, Homo sapiens
CNVNNGGCAQKCQMVRGAVQCTCHTGYRLTEDGHTC

SEQ ID NO: 29 - protein, 38 aa, Homo sapiens
CAMENGGCSHLCLRSPNPSGFSCTCPTGINLLSDGKTC

SEQ ID NO: 30 - protein, 36 aa, Homo sapiens
CGSRNGGCSHLCLPRPSGFSCACPTGIQLKGDGKTC

SEQ ID NO: 31 - protein, 22 aa, Homo sapiens
LCNGVNDCGDNSDESPQQNCRP

SEQ ID NO: 32 - protein, 42 aa, Homo sapiens
ELVFWSDVTLDRILRANLNGSNVEEVVSTGLESPGGLAVDWV

SEQ ID NO: 33 - protein, 42 aa, Homo sapiens
DKLYWTDSGTSRIEVANLDGAHRKVLLWQNLEKPRAIALHPM

SEQ ID NO: 34 - protein, 43 aa, Homo sapiens
GTIYWTDWGNTPRIEASSMDGSGRRIIADTHLFWPNGLTIDYA

SEQ ID NO: 35 - protein, 41 aa, Homo sapiens
RRMYWVDAKHHVIERANLDGSHRKAVISQGLPHPFAITVFE

SEQ ID NO: 36 - protein, 42 aa, Homo sapiens
DHVYWTDVSTDTISRAKWDGTGQEVVVDTSLESPAGLAIDWV

SEQ ID NO: 37 - protein, 42 aa, Homo sapiens
NKLYWTDAGTDRIEVANTDGSMRTVLIWENLDRPRDIVVEPM

SEQ ID NO: 38 - protein, 43 aa, Homo sapiens
GYMYWTDWGASPKIERAGMDASGRQVIISSNLTWPNGLAIDYG

SEQ ID NO: 39 - protein, 40 aa, Homo sapiens
QRLYWADAGMKTIEFAGLDGSKRKVLIGSQLPHPFGLTLY

SEQ ID NO: 40 - protein, 42 aa, Homo sapiens
ERIYWTDWQTKSIQSADRLTGLDRETLQENLENLMDIHVFHR

SEQ ID NO: 41 - protein, 42 aa, Homo sapiens
GKVYWSDSTLHRISRANLDGSQHEDIITTGLQTTDGLAVDAI

SEQ ID NO: 42 - protein, 42 aa, Homo sapiens
RKVYWTDTGTNRIEVGNLDGSMRKVLVWQNLDSPRAIVLYHE

SEQ ID NO: 43 - protein, 43 aa, Homo sapiens
GFMYWTDWGENAKLERSGMDGSDRAVLINNNLGWPNGLTVDKA

SEQ ID NO: 44 - protein, 38 aa, Homo sapiens
SQLLWADAHTERIEAADLNGANRHTLVSPVQHPYGLTL

SEQ ID NO: 45 - protein, 42 aa, Homo sapiens
GKVYYTDVFLDVIRRADLNGSNMETVIGRGLKTTDGLAVDWV

SEQ ID NO: 46 - protein, 42 aa, Homo sapiens
RNLYWTDTGRNTIEASRLDGSCRKVLINNSLDEPRAIAVFPR

SEQ ID NO: 47 - protein, 43 aa, Homo sapiens
GYLFWTDWGHIAKIERANLDGSERKVLINTDLGWPNGLTLDYD

SEQ ID NO: 48 - protein, 37 aa, Homo sapiens
RRIYWVDAHLDRIESADLNGKLRQVLVGHVSHPFALT

SEQ ID NO: 49 - protein, 36 aa, Homo sapiens
CSDFNGGCTHECVQEPFGAKCLCPLGFLLANDSKTC

SEQ ID NO: 50 - protein, 35 aa, Homo sapiens
CDILGSCSQHCYNMRGSFRCSCDTGYMLESDGRTC

SEQ ID NO: 51 - protein, 37 aa, Homo sapiens
CLENNGGCSHLCFALPGLHTPKCDCAFGTLQSDGKNC

SEQ ID NO: 52 - protein, 36 aa, Homo sapiens
CTEMPFVCSQKCENVIGSYICKCAPGYLREPDGKTC

SEQ ID NO: 53 - protein, 28 aa, Homo sapiens
CMHGGNCYFDETDLPKCKCPSGYTGKYC

SEQ ID NO: 54 - protein, 39 aa, Homo sapiens
QECDSAHFRCGSGHCIPADWRCDGTKDCSDDADEIGCAV

SEQ ID NO: 55 - protein, 41 aa, Homo sapiens
VTCQQGYFKCQSEGQCIPSSWVCDQDQDCDDGSDERQDCSQ

SEQ ID NO: 56 - protein, 39 aa, Homo sapiens
STCSSHQITCSNGQCIPSEYRCDHVRDCPDGADENDCQY

SEQ ID NO: 57 - protein, 37 aa, Homo sapiens
PTCEQLTCDNGACYNTSQKCDWKVDCRDSSDEINCTE

SEQ ID NO: 58 - protein, 37 aa, Homo sapiens
CLHNEFSCGNGECIPRAYVCDHDNDCQDGSDEHACNY

SEQ ID NO: 59 - protein, 39 aa, Homo sapiens
PTCGGYQFTCPSGRCIYQNWVCDGEDDCKDNGDEDGCES

SEQ ID NO: 60 - protein, 46 aa, Homo sapiens
HKCSPREWSCPESGRCISIYKVCDGILDCPGREDENNTSTGKYCSM

SEQ ID NO: 61 - protein, 39 aa, Homo sapiens
EQCGLFSFPCKNGRCVPNYYLCDGVDDCHDNSDEQLCGT

SEQ ID NO: 62 - protein, 39 aa, Homo sapiens
NTCSSSAFTCGHGECIPAHWRCDKRNDCVDGSDEHNCPT

SEQ ID NO: 63 - protein, 39 aa, Homo sapiens
ASCLDTQYTCDNHQCISKNWVCDTDNDCGDGSDEKNCNS

SEQ ID NO: 64 - protein, 38 aa, Homo sapiens
ETCQPSQFNCPNHRCIDLSFVCDGDKDCVDGSDEVGCV

SEQ ID NO: 65 - protein, 40 aa, Homo sapiens
LNCTASQFKCASGDKCIGVTNRCDGVFDCSDNSDEAGCPT

SEQ ID NO: 66 - protein, 41 aa, Homo sapiens
GMCHSDEFQCQEDGICIPNFWECDGHPDCLYGSDEHNACVP

SEQ ID NO: 67 - protein, 39 aa, Homo sapiens
KTCPSSYFHCDNGNCIHRAWLCDRDNDCGDMSDEKDCPT

SEQ ID NO: 68 - protein, 41 aa, Homo sapiens
FRCPSWQWQCLGHNICVNLSVVCDGIFDCPNGTDESPLCNG

SEQ ID NO: 69 - protein, 41 aa, Homo sapiens
ERCGASSFTCSNGRCISEEWKCDNDNDCGDGSDEMESVCAL

SEQ ID NO: 70 - protein, 39 aa, Homo sapiens
HTCSPTAFTCANGRCVQYSYRCDYYNDCGDGSDEAGCLF

SEQ ID NO: 71 - protein, 42 aa, Homo sapiens
RDCNATTEFMCNNRRCIPREFICNGVDNCHDNNTSDEKNCPD

SEQ ID NO: 72 - protein, 42 aa, Homo sapiens
RTCQSGYTKCHNSNICIPRVYLCDGDNDCGDNSDENPTYCTT

SEQ ID NO: 73 - protein, 40 aa, Homo sapiens
HTCSSSEFQCASGRCIPQHWYCDQETDCFDASDEPASCGH

SEQ ID NO: 74 - protein, 42 aa, Homo sapiens
RTCLADEFKCDGGRCIPSEWICDGDNDCGDMSDEDKRHQCQN

SEQ ID NO: 75 - protein, 45 aa, Homo sapiens
QNCSDSEFLCVNDRPPDRRCIPQSWVCDGDVDCTDGYDENQNCTR

SEQ ID NO: 76 - protein, 39 aa, Homo sapiens
RTCSENEFTCGYGLCIPKIFRCDRHNDCGDYSDERGCLY

SEQ ID NO: 77 - protein, 41 aa, Homo sapiens
QTCQQNQFTCQNGRCISKTFVCDEDNDCGDGSDELMHLCHT

SEQ ID NO: 78 - protein, 39 aa, Homo sapiens
PTCPPHEFKCDNGRCIEMMKLCNHLDDCLDNSDEKGCGI

SEQ ID NO: 79 - protein, 41 aa, Homo sapiens
PMCSSTQFLCANNEKCIPIWWKCDGQKDCSDGSDELALCPQ

SEQ ID NO: 80 - protein, 41 aa, Homo sapiens
RFCRLGQFQCSDGNCTSPQTLCNAHQNCPDGSDEDRLLCEN

SEQ ID NO: 81 - protein, 41 aa, Homo sapiens
HHCDSNEWQCANKRCIPESWQCDTFNDCEDNSDEDSSHCAS

SEQ ID NO: 82 - protein, 41 aa, Homo sapiens
RTCRPGQFRCANGRCIPQAWKCDVDNDCGDHSDEPIEECMS

SEQ ID NO: 83 - protein, 34 aa, Homo sapiens
EFSCKTNYRCIPKWAVCNGVDDCRDNSDEQGCEE

SEQ ID NO: 84 - protein, 40 aa, Homo sapiens
RTCHPVGDFRCKNHHCIPLRWQCDGQNDCGDNSDEENCAP

SEQ ID NO: 85 - protein, 39 aa, Homo sapiens
RECTESEFRCVNQQCIPSRWICDHYNDCGDNSDERDCEM

SEQ ID NO: 86 - protein, 39 aa, Homo sapiens
RTCHPEYFQCTSGHCVHSELKCDGSADCLDASDEADCPT

SEQ ID NO: 87 - protein, 41 aa, Homo sapiens
AYCQATMFECKNHVCIPPYWKCDGDDDCGDGSDEELHLCLD

SEQ ID NO: 88 - protein, 42 aa, Homo sapiens
VPCNSPNRFRCDNNRCIYSHEVCNGVDDCGDGTDETEEHCRK

SEQ ID NO: 89 - protein, 39 aa, Homo sapiens
KPCTEYEYKCGNGHCIPHDNVCDDADDCGDWSDELGCNK

SEQ ID NO: 90 - protein, 42 aa, Homo sapiens
QRVFWTDTVQNKVFSVDINGLNIQEVLNVSVETPENLAVDWV

SEQ ID NO: 91 - protein, 42 aa, Homo sapiens
NKIYLVETKVNRIDMVNLDGSYRVTLITENLGHPRGIAVDPT

SEQ ID NO: 92 - protein, 46 aa, Homo sapiens
GYLFFSDWESLSGEPKLERAFMDGSNRKDLVKTKLGWPAGVTLDMI

SEQ ID NO: 93 - protein, 42 aa, Homo sapiens
STIFFSDMSKHMIFKQKIDGTGREILAANRVENVESLAFDWI

SEQ ID NO: 94 - protein, 41 aa, Homo sapiens
KNLYWTDSHYKSISVMRLADKTRRTVVQYLNNPRSVVVHPF

SEQ ID NO: 95 - protein, 43 aa, Homo sapiens
GYLFFTDWFRPAKIMRAWSDGSHLLPVINTTLGWPNGLAIDWA

SEQ ID NO: 96 - protein, 42 aa, Homo sapiens
GRIFWSDATQGKTWSAFQNGTDRRVVFDSSIILTETIAIDWV

SEQ ID NO: 97 - protein, 42 aa, Homo sapiens
RNLYWTDYALETIEVSKIDGSHRTVLISKNLTNPRGLALDPR

SEQ ID NO: 98 - protein, 43 aa, Homo sapiens
HLLFWSDWGHHPRIERASMDGSMRTVIVQDKIFWPCGLTIDYP

SEQ ID NO: 99 - protein, 47 aa, Homo sapiens
GKLYWSDQGTDSGVPAKIASANMDGTSVKTLFTGNLEHLECVTLDIE

SEQ ID NO: 100 - protein, 39 aa, Homo sapiens
QKLYWAVTGRGVIERGNVDGTDRMILVHQLSHPWGIAVH

SEQ ID NO: 101 - protein, 43 aa, Homo sapiens
RYLFWADYGQRPKIERSFLDCTNRTVLVSEGIVTPRGLAVDRS

SEQ ID NO: 102 - protein, 43 aa, Homo sapiens
GYLYWADWDTHAKIERATLGGNFRVPIVNSSLVMPSGLTLDYE

SEQ ID NO: 103 - protein, 39 aa, Homo sapiens
DLLYWVDASLQRIERSTLTGVDREVIVNAAVHAFGLTLY

SEQ ID NO: 104 - protein, 42 aa, Homo sapiens
KRLYWIDTQRQVIERMFLNKTNKETIINHRLPAAESLAVDWV

SEQ ID NO: 105 - protein, 50 aa, Homo sapiens
RKLYWLDARLDGLFVSDLNGGHRRMLAQHCVDANNTFCFDNPRGLALHPQ

SEQ ID NO: 106 - protein, 43 aa, Homo sapiens
GYLYWADWGHRAYIGRVGMDGTNKSVIISTKLEWPNGITIDYT

SEQ ID NO: 107 - protein, 41 aa, Homo sapiens
DLLYWADAHLGYIEYSDLEGHHRHTVYDGALPHPFAITIFE

SEQ ID NO: 108 - protein, 42 aa, Homo sapiens
DTIYWTDWNTRTVEKGNKYDGSNRQTLVNTTHRPFDIHVYHP

SEQ ID NO: 109 - protein, 42 aa, Homo sapiens
RHIYWSDVKNKRIEVAKLDGRYRKWLISTDLDQPAAIAVNPK

SEQ ID NO: 110 - protein, 43 aa, Homo sapiens
GLMFWTDWGKEPKIESAWMNGEDRNILVFEDLGWPTGLSIDYL

SEQ ID NO: 111 - protein, 30 aa, Homo sapiens
DRIYWSDFKEDVIETIKYDGTDRRVIAKEA

SEQ ID NO: 112 - protein, 35 aa, Homo sapiens
CLHNNGGCSHICTDLKIGFECTCPAGFQLLDQKTC

SEQ ID NO: 113 - protein, 39 aa, Homo sapiens
KDCEKDQFQCRNERCIPSVWRCDEDDDCLDHSDEDDCPK

SEQ ID NO: 114 - protein, 39 aa, Homo sapiens
GTCRGDEFQCGDGTCVLAIKHCNQEQDCPDGSDEAGCLQ

SEQ ID NO: 115 - protein, 39 aa, Homo sapiens
KECEKDQFQCRNERCIPSVWRCDEDDDCLDHSDEDDCPK

SEQ ID NO: 116 - protein, 41 aa, Homo sapiens
KTCADSDFTCDNGHCIHERWKCDGEEECPDGSDESEATCTK

SEQ ID NO: 117 - protein, 28 aa, Homo sapiens
SHKCVPASWRCDGEKDCEGGADEAGCAT

SEQ ID NO: 118 - protein, 37 aa, Homo sapiens
CAPHEFQCGNRSCLAAVFVCDGDDDCGDGSDERGCAD

SEQ ID NO: 119 - protein, 44 aa, Homo sapiens
PACGPREFRCGGDGGGACIPERWVCDRQFDCEDRSDEAAELCGR

SEQ ID NO: 120 - protein, 40 aa, Homo sapiens
AACATVSQFACRSGECVHLGWRCDGDRDCKDKSDEADCPL

SEQ ID NO: 121 - protein, 46 aa, Homo sapiens
NRIYWCDLSYRKIYSAYMDKASDPKEQEVLIDEQLHSPEGLAVDWV

SEQ ID NO: 122 - protein, 46 aa, Homo sapiens
NRIYWCDLSYRKIYSAYMDKASDPKEREVLIDEQLHSPEGLAVDWV

SEQ ID NO: 123 - protein, 42 aa, Homo sapiens
KHIYWTDSGNKTISVATVDGGRRRTLFSRNLSEPRAIAVDPL

SEQ ID NO: 124 - protein, 43 aa, Homo sapiens
GFMYWSDWGDQAKIEKSGLNGVDRQTLVSDNIEWPNGITLDLL

SEQ ID NO: 125 - protein, 43 aa, Homo sapiens
QRLYWVDSKLHQLSSIDFSGGNRKTLISSTDFLSHPFGIAVFE

SEQ ID NO: 126 - protein, 42 aa, Homo sapiens
DKVFWTDLENEAIFSANRLNGLEISILAENLNNPHDIVIFHE

SEQ ID NO: 127 - protein, 4 aa, Homo sapiens
YWTD